(19) United States
(12) Patent Application Publication
(10) Pub. No.: US 2022/0137034 A1
(43) Pub. Date: May 5, 2022

(54) DETECTION AND QUANTIFICATION OF SMALL MOLECULES

(71) Applicant: Aarhus Universitet, Aarhus C (DK)

(72) Inventors: Kurt Vesterager Gothelf, Risskov (DK); Michael Rosholm Mortensen, Zürich (CH); Malthe Hansen-Bruhn, Højbjerg (DK); Line Debois Frejlev Nielsen, Aarhus C (DK); Jesper Vinther Sørensen, Åbyhøj (DK)

(21) Appl. No.: 17/310,792

(22) PCT Filed: Feb. 21, 2020
(86) PCT No.: PCT/EP2020/054587

§ 371 Date: Aug. 24, 2021

(30) Foreign Application Priority Data
Feb. 25, 2019 (EP) 19159127.0

(51) Int. Cl. G01N 33/542; G01N 21/64

(52) U.S. Cl. CPC G01N 33/542; G01N 2021/6439; G01N 2458/10; G01N 21/6428

(57) ABSTRACT

The present invention relates to an in vitro method for determining the presence, absence and/or concentration of an analyte in a sample. The method uses an optically based competition assay comprising a labelled analyte binding protein and a labelled analyte analogue. The concentration/presence of the analyte is determined by inhibitory binding of the analyte to the analyte binding protein thereby impeding binding of the analyte analogue to the analyte binding protein. The invention further relates to kits, solid supports, cartridges, detection chips and uses thereof.

### TECHNICAL FIELD OF THE INVENTION

The present invention relates to an in vitro method for determining the presence, absence and/or concentration of an analyte in a sample. In particular, the present invention relates to a method using a fluorescently based competition assay comprising a fluorescently labelled analyte binding protein and a fluorescently labelled analyte analogue.

### BACKGROUND OF THE INVENTION

The quantification of small molecule analytes in particular in complex biological samples such as blood is of importance e.g. for determining the concentration of a specific drug molecule in the blood of patients. Traditionally this is done by pre-treatment of the blood sample and analysis by HPLC-MS in clinical biochemical departments. This is however time consuming, require specialists and require sophisticated equipment.

Few point-of-care (POC) technologies are available for the detection of small molecules, where the glucose meter is an outstanding exception, which relies on the enzymatic conversion of glucose.

For almost all small molecule drugs, such enzymatic conversion is not available and other techniques must be applied.

Surface plasmon resonance (SPR) is an assay principle that has been employed in a variety of setups for small molecule detection. Surface plasmon resonance is a heterogeneous assay type that utilizes mass changes at a surface caused by binding, usually as competitive immunoassays. The setup allows for detection of a wide range of compounds at the relevant ranges, however it is an expensive technology. (Sensors 2010, 10, 7323-7346).

The proximity hybridisation technique, usually used for detection of compounds exhibiting multiple epitopes, was recently adapted for small molecule detection. Employing the bivalent binding interaction of antibodies allowed for detection of digoxin in the nanomolar range. The assay exhibited detection times of 10-30 mins. (Anal. Chem. 2018, 90, 9667-9672)

Another type of detection system which is used for small molecule detection is the so-called quench body system (Anal. Methods, 2016, 8, 7774-7779), where a fluorophore close to the binding site is in part quenched in the absence of the analyte. This is not a ratiometric system and furthermore it must be optimized significantly for each analyte.

Ricci and co-workers displayed a sensor in which structural change of DNA induced by proximity hybridisation allowed for detection of antibodies in 10% plasma in a homogenous fashion (J. Am. Chem. Soc. 2018, 140, 947-953).

Plaxco and coworkers showed measurement of small molecule targets in the μM range directly in undiluted whole blood with overall good precision employing a heterogenous aptamer based sensor system. The setup has likewise been implemented directly in live animals for real time measurement of small molecule targets. The real time measurement allowed for precise control of drug in the animal model based on feedback-controlled dosing (Proc. Natl. Acad. Sci. USA 2017, 114, 645-650).

In recent work a homogenous for detection of cortisol showed drastic improvement relative to the conventional heterogeneous cortisol assay types. The work was implemented in a Point of care pchip for single molecule array measurements with vastly increased sensitivity compared to conventional methods (J. Am. Chem. Soc. 2018, 140, 18132-18139).

In a series of papers and a patent Kai Johnsson has described semisynthetic protein peptide systems that provides a FRET readout upon small molecule binding in solution (J. Am. Chem. Soc. 2009, 131, 5873; J. Am. Chem. Soc., 2009, 131, pp 5873-5884; Angew. Chem. Int. Ed. 2014, 53, 1302, WO2015067302A1).

A so-called strand displacement assay for detection of small molecules has also been developed. (J. Am. Chem. Soc. 2014, 136, 11115-11120; EP2895617A1). A platform for digoxin detection within the relevant range, employing a highly selective assay and a recyclable and passively driven G-chip has also been developed. (Adv. Sci. 2019, doi/10.1002/advs.201802051). Furthermore, a rapid amplification based homogenous assay for detection of Methotrexate in human plasma within 4 minutes within the relevant ranges has been developed (ACS Sens. 2018, 3, 9, 1706-1711).

Most of the assays described above have, however, not been developed for detection of analytes in blood. In the few examples where this is the case the assay is not generic, too slow or not suitable for a POCT device.

Hence, an improved method for fast determination of an analyte would be advantageous, and in particular a more efficient and/or reliable method for fast determination of the concentration of an analyte in a blood sample would be advantageous.

### SUMMARY OF THE INVENTION

The present invention relates to an in vitro method for determining the presence, absence and/or concentration of an analyte (1) in a sample. The method uses an optically based competition assay comprising a optically labelled analyte binding protein (3) and a optically labelled analyte analogue (6). The concentration/presence of the analyte (1) is determined by inhibitory binding of the analyte (1) to the analyte binding protein (3) thereby impeding binding of the analyte analogue (6) to the analyte binding protein (3). The invention further relates to kits, solid supports (7), cartridges (8), detection chips (10) and uses thereof. The method is schematically outlined in FIG. 1, whereas the solid support, cartridge and detection chip are outlined in FIG. 3.

Thus, an object of the present invention relates to the provision of a sensitive method for determining the presence, and in particular the concentration, of an analyte in a sample. In particular, it is an object of the present invention to provide a method that solves the problems of the prior art with fast determination of the concentration of an analyte in a sample.

Thus, one aspect of the invention relates to an (in vitro) method for determining the presence and/or concentration of an analyte (1) in a sample (2), said method comprising
I. having provided a sample (2) to be analyzed for the presence of the analyte (1);
II. providing an analyte binding protein (3), said analyte binding protein comprising
   a binding site (such as a paratope) (4) for the analyte (1); and
   a covalently linked first member (5A) of an optical signal pair (5);
III. providing an analyte analogue (6), said analyte analogue (6) being covalently coupled to a second member (5B) of the optical signal pair (5);
   wherein, when said analyte binding protein (3) and said analyte analogue (6) are in proximity, an optical signal is generated which is different from the optical signal generated when the analyte binding protein (3) and the analyte analogue (6) are not in proximity, such as when the analyte (1) is present;
IV. bringing in contact said sample (2), said analyte binding protein (3) and said analyte analogue (6), and
V. determining the presence or absence of the analyte (1) in said sample (2) by measuring an optical signal;
   and/or
VI. determining the concentration of the analyte (1) in said sample (2) by measuring an optical signal.

In a preferred embodiment, the first member (5A) of the optical signal pair (5) is covalently coupled to the analyte binding protein (3) through a first oligonucleotide linker (preferably a DNA linker) and the analyte analogue (6) is covalently coupled to the second member (5B) of the optical signal pair (5) through a second oligonucleotide linker (preferably a DNA linker). This setup is tested in the example section.

In another preferred embodiment, said sample (2) is brought in contact with the analyte binding protein (3) before being brought in contact with said analyte analogue (6). Example 11 documents that the order of mixing the assay species influences on the efficiency of the assay.

Another aspect of the present invention relates to relates to a kit comprising
I. a first vial comprising an analyte binding protein (3), said analyte binding protein comprising
   a binding site (4) for an analyte (1); and
   a covalently linked first member (5A) of an optical signal pair (5);
II. a second vial comprising an analyte analogue (6), said analyte analogue (6) being covalently coupled to a second member (58) of the optical signal pair (5);
III. optionally, a (porous) solid support material (7) for depositing the content of the first vial and the second vial at different (indicated) distinct regions; and
IV. optionally, a cartridge (8) for receiving the porous solid support material (7).

Yet another aspect of the present invention is to provide a porous solid support comprising
a) a first region comprising an analyte binding protein (3), said analyte binding protein (3) comprising
   a binding site (4) for an analyte (1); and
   a covalently linked first member (5A) of a fluorescent pair (5);
b) a second region comprising an analyte analogue (6), said analyte analogue (6) being covalently coupled to a second member (58) of the fluorescent pair (5).

In a preferred embodiment, the solid support is configured for receiving a sample, and bringing said sample in contact with the analyte binding protein (3) before bringing said sample in contact with said analyte analogue (6).

A further aspect of the invention relates to a cartridge (8):
for mounting of the porous solid support (7) according to the invention; and
containing a detection chamber (9) which is made of an optically transparent material allowing for optical determination of the analyte (1) in the sample (2).

Yet a further aspect relates to a detection chip (10) comprising the solid support (8) according to the invention mounted in a cartridge (8) according to the invention.

Still another aspect of the present invention is to provide the use of the kit according to the invention and/or the porous solid support (7) according to the invention and/or the cartridge (8) according to the invention and/or the detection chip (10) for determining the presence, absence and/or concentration of an analyte (1) in a sample (2).

### BRIEF DESCRIPTION OF THE FIGURES

FIG. 1 illustrates the concept of the assay, and shows fluorescent data and a dose response curve for detection of Dabigatran. A) The analyte binding protein (3) is covalently linked to the first member (5A) of the optical pair. When the sample (2) is added, one of two things can happen. If the sample (2) contains analyte (1) then the paratope (4) will be occupied by analyte. If the sample (2) does not contain analyte (1) then the paratope (4) is unoccupied. In absence of analyte (1), the analyte analogue (6) modified with the second member (5B) of the optical pair (5) is bound by the paratope (4) producing a signal. In presence of analyte (4) the analyte analogue (6) is unable to bind the paratope (4) thereby resulting in no signal. B) The resulting change in fluorescent spectra induced by analyte concentrations. C) Dose response curve of the assay at different analyte concentrations.

FIG. 2 illustrates the formation of the analyte binding protein (3). To the paratope of a protein is guided an activated group (N-hydroxysuccinimide ester) onto which the first member (5A) of the optical pair (5) is covalently linked (reactive strand (13)). The guiding motif is the analyte conjugated to a DNA strand fully complementary to the activated DNA strand (guiding strand (11)). Upon addition of a releasing strand (12) complementary to the guiding strand (11), the functional analyte binding protein (3) is generated.

FIG. 3 depicts the detection chip with the various components. The detection chip (10) consists of a cartridge (8) fitted with a solid support (7) onto which the assay reagents are dried at independent regions. The solid support (7) concludes in a terminal readout window (detection chamber) (9) in which the optical readout is performed.

FIG. 4 illustrates the structure of the small molecule analytes. A) dabigatran B) linezolid and C) apixaban.

FIG. 5 shows the performance of the Dabigatran assay in a fluorometer setup in both buffer and plasma. A) The dose response of the assay performed in HEPES (pH 7, 50 mM) resulting in decrease in signal up to 2 eq. Dabigatran. B) Presence and absence of dabigatran in 87% plasma is readily distinguished within 10 min. C) Absence of dabigatran results in rapid signal generation in 87% plasma. D) Presence of dabigatran results in no signal over 10 min in 87% plasma.

FIG. 6 illustrates the dose response of the Dabigatran assay in plasma and whole blood, the test against the HEMOCLOT and detection in other complex matrices. A) In the detection chip (10) is the analyte dabigatran in both whole blood (90%, dashed line) and plasma (85%, solid line) readily quantified within the range 0-100 ng/mL. B) The presence (1 μM) and absence of dabigatran is determined in complex matrices (90%) such as milk, urine, beer and saliva in addition to plasma. C) The detection chip (10) fitted with the dabigatran assay (prototype, solid line, 90% whole blood) is tested against the HEMOCLOT method (dashed line) for dabigatran measurement and performs similarly.

FIG. 7 shows the kinetic performance of the Apixaban assay in a fluorometer setup and the dose response in plasma employing the detection chip (10). A) absence of apixaban resulting in rapid signal in 87% plasma. B) Presence of apixaban results in only minor signal over 10 min in 87% plasma. C) In the detection chip (10) does the presence of apixaban from 0-500 nM result in a linear response in plasma.

FIG. 8 shows the kinetic performance of the linezolid assay with both single and double modified analyte binding protein (3) in a fluorometer setup and the dose response in both plasma and whole blood employing the detection chip (10). A) absence of linezolid resulting in rapid signal in 87% plasma using the single modified analyte binding protein (3). B) Presence of linezolid results in only minor signal generation over 10 min in 87% plasma using the single modified analyte binding protein (3). C) absence of linezolid resulting in rapid signal in 79% plasma using the double modified analyte binding protein (3). D) Presence of linezolid results in only minor signal generation over 10 min in 79% plasma using the double modified analyte binding protein (3). E) In the detection chip (10) does the presence of linezolid from 0-2000 nM result in a decrease in FRET signal in both 93% whole blood (dashed line) and 93% plasma (solid line).

FIG. 9 shows the performance of the dabigatran assay and controls with unspecifically labelled protein (Global Cy3 Conjugate (Glo-Cy3) and Global DNA Conjugate (Glo-DNA)) in a fluorometer setup at 25 nM. The signal difference is between presence and absence of 10 eq. dabigatran after 10 min. in HEPES pH 7.5 buffer employing the different protein conjugates on a fluorometer setup is shown. The analyte binding protein (3) performs significantly better than the controls Global Cy3 Conjugate (Glo-Cy3) and Global DNA Conjugate (Glo-DNA).

FIG. 10 shows the performance of the analyte binding protein (3) (dabigatran) assay and controls with unspecifically labelled protein (Global Cy3 Conjugate (Glo-Cy3) and Global DNA Conjugate (Glo-DNA)) employing the detection chip (10) and analyte analogue (6) (dabigatran). A) The signal difference between presence and absence of 1 μM dabigatran after 10 min. in 100% plasma is shown. The analyte binding protein (3) performs significantly better than Global DNA Conjugate (Glo-DNA). B) The raw spectrum of the Global Cy3 Conjugate (Glo-Cy3) after 10 min. in 100% plasma in the absence and presence of 1 μM dabigatran employing the detection chip (10). In the background spectrum no protein conjugate is present in the window, thus only analyte analogue (6) signal is seen. C) The raw spectrum of the analyte binding protein (3) (dabigatran) assay after 10 min. in 100% plasma in absence and presence of 1 μM dabigatran employing the detection chip (10) and analyte analogue (6) (dabigatran). Distinct peaks from the optical pair (5) is clearly observed.

FIG. 11 shows the performance of the analyte binding protein (3) (dabigatran) assay in a competition experiment in HEPES pH 7.5 buffer in a fluorometer setup at 25 nM. The percentile change in FRET is depicted compared to the initial first measurement (T=0 min). A) The absence of 10 eq. dabigatran allows signal to rapidly evolve. If the analyte binding protein (3) has been saturated with 10 eq. dabigatran prior to addition of the analyte analogue (6), no change in signal over time is obtained. Likewise, if the analyte binding protein (3) has been saturated with 1 eq. the analyte analogue (6) prior to addition of 10 eq. dabigatran, no change in signal over time is obtained. B) The absence of 1 eq. dabigatran allows signal to rapidly evolve. If the analyte binding protein (3) has been saturated with 1 eq. dabigatran prior to addition of the analyte analogue (6), only minor change in signal is obtained. Likewise, if the analyte binding protein (3) has been saturated with 1 eq. the analyte analogue (6) prior to addition of 1 eq. dabigatran, only minor change in signal is obtained.

The present invention will now be described in more detail in the following.

### DETAILED DESCRIPTION OF THE INVENTION

### Definitions

Prior to discussing the present invention in further details, the following terms and conventions will first be defined:

Small Molecules

In the Present Context, the Term “Small Molecule” Relates to a Low Molecular weight (<900 daltons) organic compound that may regulate a biological process, with a size on the order of 1 nm. Most drugs are small molecules. Larger structures such as nucleic acids and proteins, and many polysaccharides are not small molecules.

Antibody

The term “antibody” as used herein refers to a protein of the immunoglobulin (Ig) superfamily that binds non-covalently to certain substances (antigens/analytes) to form an antibody-antigen/analyte complex. Antibodies can be endogenous, or polyclonal wherein an animal is immunized to elicit a polyclonal antibody response or by recombinant methods resulting in monoclonal antibodies produced from hybridoma cells or other cell lines. It is understood that the term “antibody” as used herein includes within its scope any of the various classes or sub-classes of immunoglobulin derived from any of the animals conventionally used.

Antibody Fragments

The term “antibody fragments” as used herein refers to fragments of antibodies that retain the principal selective binding characteristics of the whole antibody. Particular fragments are well-known in the art, for example, Fab, Fab′, and F(ab′)₂ which are obtained by digestion with various proteases, pepsin or papain, and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment, which consists of a VH domain; (iii) isolated CDR regions; and (iv) single-chain Fv molecules (scFv) described above. In addition, arbitrary fragments can be made using recombinant technology that retains antigen-recognition characteristics.

Analyte

The term “analyte” as used herein refers to any entity that an analyte binding protein has affinity for.

Analyte Analogue

The term “analyte analogue” as used herein refers to an analyte that has been modified to contain a reporter molecule and optionally to alter the affinity of the analyte analogue for the analyte binding protein, compared to an unmodified analyte.

Analyte Binding Protein

The term “analyte binding protein” as used herein refers to a protein that has affinity for a discrete epitope, antigen or analyte that can be used with the methods of the present invention. Preferably, the analyte binding protein is an antibody or fragment thereof.

Affinity

The term “affinity” as used herein refers to the strength of the binding interaction of two molecules, such as an antibody and its antigen (or the analyte binding protein and the analyte/analyte analogue according to the invention). For bivalent molecules such as antibodies, affinity is typically defined as the binding strength of one binding domain for the antigen, e.g. one Fab fragment for the antigen. The binding strength of both binding domains together for the antigen is referred to as “avidity”. As used herein “High affinity” refers to a ligand that binds to an antibody having an affinity constant (Ka) greater than 10⁴ M⁻¹, typically 10⁵-10¹¹ M⁻¹; as determined by inhibition ELISA or an equivalent affinity determined by comparable techniques such as, for example, Scatchard plots or using K_{d}/dissociation constant, which is the reciprocal of the Kₐ, etc.

Energy Transfer

The term “energy transfer” as used herein refers to the process by which the excited state energy of an excited group, e.g. fluorescent reporter dye, is conveyed through space or through bonds to another group, e.g. a quencher moiety or fluorophor, which may attenuate (quench) or otherwise dissipate or transfer the energy to another reporter molecule or emit the energy at a longer wavelength. Energy transfer typically occurs through fluorescence resonance energy transfer (FRET).

Optical Pair

The term “optical pair” as used herein refers to any two moieties that can form a pair allowing for optical determination of such a pair. A pair could be to parts of a protein pair, which are only functional when they are in proximity. An example of such a pair is Cy3 and Cy5.
I. Other preferred optical pairs are fluorescent pairs. a FRET pair such as Cy3 and Cy5, Cy5.5 and Cy7, Atto425 and Atto 520, Atto488 and Atto590, Atto488 and Atto647N, Atto488 and Atto550, Atto488 and Atto565, Atto488 and Atto655, Atto550 and Atto 647N, Atto323 and Atto647N, Atto532 and Atto655, Atto550 and Atto655, Atto550 and Atto590, Atto550 and Atto647N, Atto565 and Atto590, Atto565 and Atto647N, Atto590 and Atto655, Atto590 and Atto620, Atto590 and Atto647N, Atto590 and Atto680, Atto620 and Atto680, Alexa Fluor 350 and Alexa Fluor 488, Alexa Fluor 488 and Alexa Fluor 546, Alexa Fluor 488 and Alexa Fluor 555, Alexa Fluor 488 and Alexa Fluor 568, Alexa Fluor 488 and Alexa Fluor 594, Alexa Fluor 488 and Alexa Fluor 647, Alexa Fluor 546 and Alexa Fluor 568, Alexa Fluor 546 and Alexa Fluor 594, Alexa Fluor 546 and Alexa Fluor 647, Alexa Fluor 555 and Alexa Fluor 594, Alexa Fluor 555 and Alexa Fluor 647, Alexa Fluor 568 and Alexa Fluor 647, Alexa Fluor 594 and Alexa Fluor 647, TMR and Texas Red, Texas Red and Cy5, FAM and Cy5, FAM and TAMRA, preferably the FRET pair is Cy3 and Cy5;
II. a fluorophore-quencher pair such as Alexa Fluor 350 and QSY 35, Alexa Fluor 350 and dabcyl, Alexa Fluor 350 and BHQ-0, Alexa Fluor 350 and TQ1, Alexa Fluor 350 and TQ2, Alexa Flour 350 and BHQ-1, Alexa Fluor 488 and QSY 35, Alexa Fluor 488 and dabcyl, Alexa Fluor 488 and QSY 7&9, Alexa Fluor 488 and BHQ-0, Alexa Fluor 488 and BHQ-1, Alexa Fluor 488 and TQ1, Alexa Fluor 488 and TQ2, Alexa Fluor 488 and TQ3, Alexa Fluor 546 and QSY 35, Alexa Fluor 546 and dabcyl, Alexa Fluor 546 and QSY 7&9, Alexa Fluor 546 and BHQ-2, Alexa Fluor 546 and BHQ-1, Alexa Fluor 546 and BHQ-3, Alexa Fluor 546 and TQ2, Alexa Fluor 546 and TQ3, Alexa Fluor 546 and TQ4, Alexa Fluor 546 and QSY 21, Alexa Fluor 555 and QSY 7&9, Alexa Fluor 555 and BHQ-2, Alexa Fluor 555 and BHQ-1, Alexa Fluor 555 and QSY 21, Alexa Fluor 555 and BHQ-3, Alexa Fluor 555 and TQ2, Alexa Fluor 555 and TQ3, Alexa Fluor 555 and TQ4, Alexa Fluor 568 and QSY 7&9, Alexa Fluor 568 and BHQ-2, Alexa Fluor 568 and QSY 21, Alexa Fluor 594 and QSY 21, Alexa Fluor 594 and QSY 7&9, Alexa Fluor 594 and BHQ-2, Alexa Fluor 594 and BHQ-3, Alexa Fluor 594 and TQ3, Alexa Fluor 594 and TQ4, Alexa Fluor 594 and TQS, Alexa Fluor 647 and QSY 21, Alexa Fluor 647 and BHQ-3, Alexa Fluor 647 and TQ4, Alexa Fluor 647 and TQS, Alexa Fluor 647 and TQ6, Alexa Fluor 680 and TQS, Alexa Fluor 680 and TQ6, Alexa Fluor 680 and TQ7, Alexa Fluor 750 and TQ6, Alexa Fluor 750 and TQ7, Cy3 and BHQ-1, Cy3 and BHQ-2, Cy3 and BHQ-3, Cy3 and QSY 7&9, Cy3 and QSY 21, Cy3 and TQ2, Cy3 and TQ3, Cy3 and TQ4, Cy3 and Eclipse Dark Quencher, Cy3.5 and BHQ-2, Cy3.5 and Deep Dark Quencher II, Cy5 and BHQ-3, Cy5 and QSY 21, Cy5 and BHQ-2, Cy5 and BHQ-1, Cy5 and TQ4, Cy5 and TQ5, Cy5 and TQ6, Cy5 and Deep Dark Quencher II, Cy5.5 and BHQ-3, Cy5.5 and QSY 21, Cy5.5 and TQ5, Cy5.5 and TQ6, Cy5.5 and TQ7, Cy7 and TQ6, Cy7 and TQ7, TAMRA and BHQ-1, TAMRA and BHQ-2, TAMRA and QSY 7&9, TAMRA and dabcyl, FAM and BHQ-1, FAM and BHQ-2, FAM and BHQ-0, FAM and QSY 7&9, FAM and dabcyl, FAM and TQ1, FAM and TQ2, FAM and TQ3, Texas Red and BHQ-2, Texas Red and BHQ-1, Texas Red and BHQ-3, Texas Red and QSY 7&9, Texas Red and QSY 21, Texas Red and TQ3, Texas Red and TQ4, Texas Red and TQ5, Texas Red and dabcyl, TET and BHQ-1, TET and BHQ-2, ATT0488 and BHQ-0, ATT0532 and BHQ-1, ATT0532 and Eclipse Dark Quencher, ATT0550 and BHQ-2, ATT0550 and BHQ-1, ATT0550 and BHQ-3, ATT0550 and TQ2, ATT0550 and TQ3, ATT0550 and TQ4, ATT0550 and QSY 7&9, ATT0550 and QSY 21, ATT0550 and Deep Dark Quencher II, ATT0565 and BHQ-2, ATT0565 and BHQ-1, ATT0565 and BHQ-3, ATT0565 and TQ2, ATT0565 and TQ3, ATT0565 and TQ4, ATT0565 and QSY 7&9, ATT0565 and QSY 21, ATT0565 and Deep Dark Quencher II, ATT0590 and BHQ-2, ATT0590 and Deep Dark Quencher II, ATT0620 and BHQ-2, ATT0620 and Deep Dark Quencher II, ATT0620 and BHQ-3, HEX and TQ2, HEX and TQ3, HEX and TQ4, HEX and BHQ-1, HEX and BHQ-2, HEX and BHQ-3, HEX and QSY 7&9, HEX and QSY 21, TET and TQ2, TET and TQ3, TET and TQ4, TET and BHQ-1, TET and BHQ-2, TET and BHQ-3, TET and QSY 7&9, TET and QSY 21, JOE and TQ2, JOE and TQ3, JOE and TQ4, JOE and BHQ-1, JOE and BHQ-2, JOE and BHQ-3, JOE and QSY 7&9, JOE and QSY 21.

Fluorescent Pair

The term “fluorescent pair” as used herein refers to any two moieties that participate in energy transfer. Typically, one of the moieties acts as a fluorescent reporter, i.e. donor, and the other acts as an acceptor, which may be a quenching compound or a compound that absorbs and re-emits energy in the form of a fluorescent signal.

Quencher

The term “quencher” or “quenching moiety” as used herein refers to a compound that is capable of absorbing energy from an energy donor that is not re-emitted (non-fluorescent) or re-emitted at a detectably different wavelength from the energy emitted by the donor molecule. In this respect, quenchers may be essentially non-fluorescent or fluorescent.

Sample

The term “sample” as used herein refers to any material that may contain an analyte of interest. Typically, the sample comprises a population of cells, cellular extract, subcellular components, tissue culture, a bodily fluid, tissue, and reaction mixtures. The sample may be in an aqueous solution, a viable cell culture or immobilized on a solid or semi-solid surface such as a gel, a membrane, a glass surface, a microparticle or on a microarray. Preferably, the sample is a blood sample, such as a blood plasma sample.

Kit

The term “kit” as used herein refers to a packaged set of related components, typically one or more compounds or compositions.

Method for Determining the Presence and/or Concentration of an Analyte in a Sample

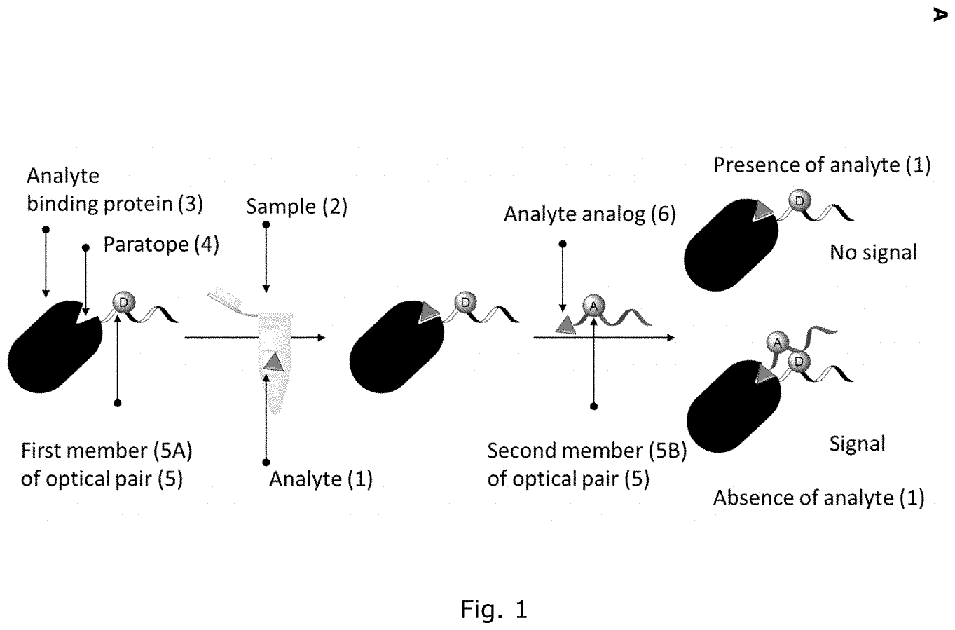

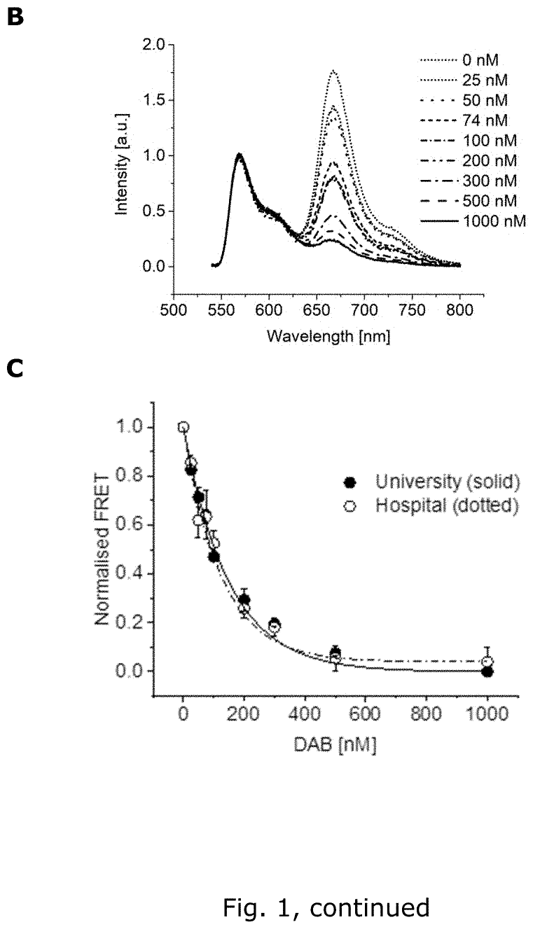

A described above, the present invention relates to a method where the concentration of an analyte can be determined in a sample, preferably a blood plasma sample. As outlined in FIG. 1, the method is a fluorescent-based competition assay where an analyte and s fluorescently labelled analyte analogue competes in the binding to a fluorescently labelled analyte binding protein.

Preferably, the method is based on FRET technology. The method further takes advantage of site-directed labelling of the analyte binding protein making the use of FRET very efficient. Thus, an aspect of the invention relates to an (in vitro) method for determining the presence and/or concentration of an analyte (1) in a sample (2), said method comprising
I. having provided a sample (2) to be analyzed for the presence of the analyte (1);
II. providing an analyte binding protein (3), said analyte binding protein comprising
   a binding site (such as a paratope) (4) for the analyte (1); and
   a covalently linked first member (5A) of an optical signal pair (5);
III. providing an analyte analogue (6), said analyte analogue (6) being covalently coupled to a second member (58) of the optical signal pair (5);
   wherein, when said analyte binding protein (3) and said analyte analogue (6) are in proximity, an optical signal is generated which is different from the optical signal generated when the analyte binding protein (3) and the analyte analogue (6) are not in proximity, such as when the analyte (1) is present;
IV. bringing in contact said sample (2), said analyte binding protein (3) and said analyte analogue (6), and
V. determining the presence or absence of the analyte (1) in said sample (2) by measuring an optical signal;
   and/or
VI. determining the concentration of the analyte (1) in said sample (2) by measuring an optical signal.

A special feature of the method of the invention is that it can determine the concentration of the analyte in the sample. Thus, in a preferred embodiment, the concentration of the analyte in the sample is determined. Concentration determination of different analytes are further described in examples 3-8.

In a preferred embodiment, the first member (5A) of the optical signal pair (5) is covalently coupled to the analyte binding protein (3) through a first oligonucleotide linker (preferably a DNA linker) and the analyte analogue (6) is covalently coupled to the second member (58) of the optical signal pair (5) through a second oligonucleotide linker (preferably a DNA linker). This setup is used in the example section. The use of the oligonucleotide linkers also makes it possible to make specific labelling instead of random labelling. This is shown in e.g. examples 9 and 10.

In another preferred embodiment, said sample (2) is brought in contact with the analyte binding protein (3) before being brought in contact with said analyte analogue (6). Example 11 documents that the order of mixing the assay species influences on the efficiency of the assay.

To determine the presence or absence of an analyte, it may be advantageous to compare the fluorescent to one or more reference levels. Thus, in another embodiment, in said determination step V.,
when said generated fluorescent signal is different from a reference fluorescent signal, it is indicative of the presence of said analyte (1) in said sample (2); OR
when said generated fluorescent signal is not different from a reference fluorescent signal, it is indicative of the absence of the analyte (1) in said sample (2).

The method of the invention is based on a change in signal when an analyte (1) binds to the labelled analyte binding protein (3), thereby releasing the labelled analyte analogue (6). Thus, in a further embodiment, in said determination step V. and/or VI., the determination is performed by illuminating the sample (2) with an appropriate wavelength and observing the sample (2) at relevant wavelengths, wherein the sample (2) generates a change in detectable signal in the presence of the analyte (1) in the sample (2). It is noted that such effect can preferably be obtained e.g. using a FRET pair or a fluorophore-quencher pair. In the example section, FRET is used.

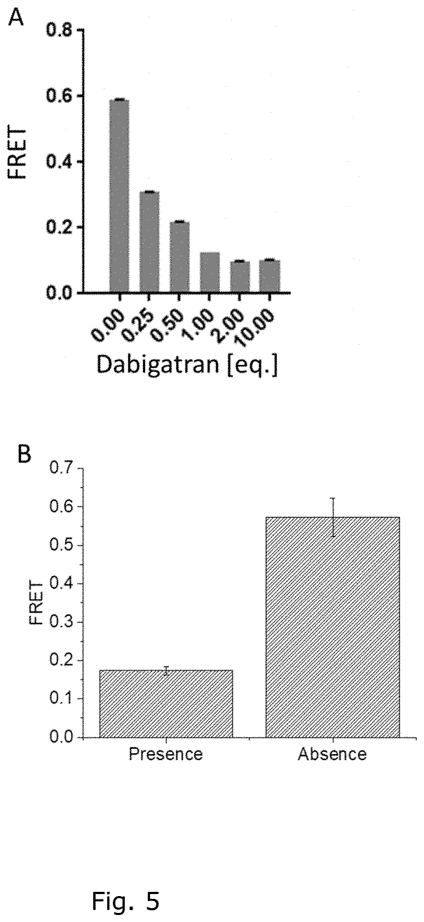

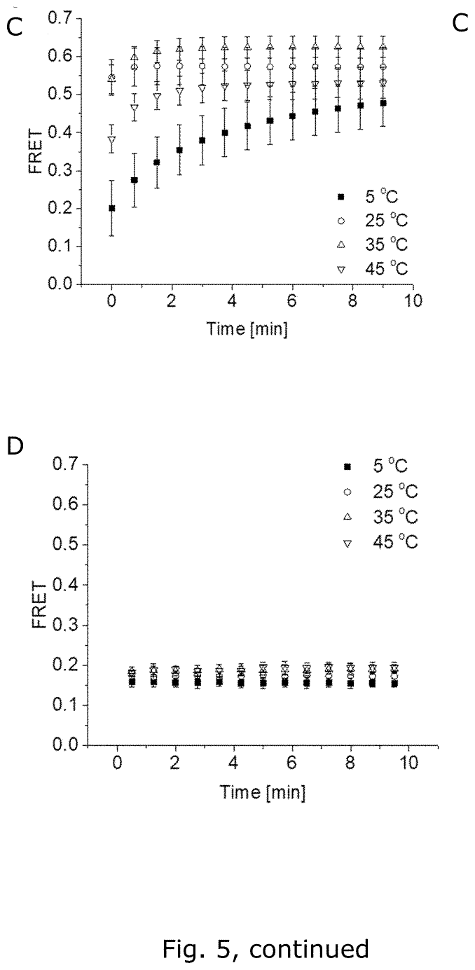

When determining the concentration in a sample other reference levels may be used. Thus, in an embodiment, in said determination step VI., a reference level is a subset of known concentrations, such as obtained from a titration curve of the analyte. Examples 3-8+FIGS. 5-8 show such titration curves of known concentrations for the analytes dabigatran, linezolid and apixaban.

The sample (2) to be analyzed for the presence of the analyte in question may be from different sources. Thus, in an embodiment said sample (2) is a biological sample, a water sample, environmental sample, a food sample, a beverage, a surface swap, a medical formulation, a drug formulation, an addictive substance or formulation. Medical formulations may be dissolved or diluted. A medical formulation could also be an illegal drug.

In yet an embodiment, said biological sample (2) has been obtained from a human or animal, such as a mammal. The sample may have been previously obtained from a subject, meaning that the method is an in vitro method. In yet a further embodiment said biological sample (2) is selected from the group consisting of a blood sample, such as whole blood, such as blood plasma or blood serum, saliva, urine, CSF and a tissue sample.

The analyte to be determined may be of different types. Thus, in an embodiment, said analyte (1) is selected from the group consisting of a small molecule, a peptide and a protein, preferably a small molecule. In yet an embodiment said small molecule has a molecular weight of less than 900 Dalton, such as in the range 100-900, 200-900, 300-900, 400-900 or 500-900 Daltons. In a more specific embodiment, said small molecule is selected from the group consisting of an anticoagulant, such as Dabigatran and Apixaban, an antibiotic, such as such as Linezolid, a drug, such as an anti-cancer drug. Data for these compounds are presented in examples 3-8.

In a further embodiment, said analyte (1) is selected from the group consisting of anticoagulants such as Warfarin, Edoxaban, Rivaroxaban and Betrixaban, and immusupressants such as Methotrexate, Cyclosporine, Tacrolimus, Sirolimus, and Everolimus, and illegal drugs such as Cannaboids, Cocaine, Opiates (Heroin), Methamphetamine, Amphetamine, and Phencyclidine.

In yet a further embodiment, said analyte (1) is selected from the groups consisting of:

1)

Drugs such as selected from the group consisting of Atorvastatin, Levothyroxine, Lisinopril, Omeprazole, Metformin, Amlodipine, Simvastatin, Metoprolol, Losartan, Azithromycin, Zolpidem, Hydrochlorothiazide, Furosemide, Metoprolol, Pantoprazole, Gabapentin, Amoxicillin, Prednisone, Sertraline, Tamsulosin, Fluticasone, Pravastatin, Tramadol, Montelukast, Escitalopram, Carvedilol, Alprazolam, Warfarin, Meloxicam, Clopidogrel, Amoxicillin, Allopurinol, Bupropion, Lisinopril, Citalopram, Losartan, Atenolol, Cialis, Duloxetine, Fluoxetine, Fenofibrate, Crestor, Venlafaxine, Ventolin, Cyclobenzaprine, Trazodone, and Methylprednisolone.

2)

(Illegal) food additives, such as selected from the group consisting of cinnamyl anthranilate, cobalt salts, coumarin, cyclamate, diethyl pyrocarbonate (DEPC), dulcin (p-ethoxy-phenylurea), monochloroacetic acid, nordihydroguaiaretic acid (NDGA), oil of calamus, polyoxyethylene-8-stearate (Myrj 45), safrole, thiourea, and melamine.

3)

Vitamins such as selected from the group consisting of all-trans-Retinol, Retinals, and alternative provitamin A-functioning Carotenoids including all-trans-beta-carotene, Thiamine, Riboflavin, Niacin, Niacinamide, Nicotinamide riboside, Pantothenic acid, Pyridoxine, Pyridoxamine, Pyridoxal, Biotin, Folates, Folic acid, Cyanocobalamin, Hydroxocobalamin, Methylcobalamin, Adenosylcobalamin, Ascorbic acid, Cholecalciferol (D3), Ergocalciferol (D2), Tocopherols, Tocotrienols, Phylloquinone, and Menaquinones.

4)

Exogenous Anabolic Androgenic Steroids (AAS), such as selected from the group consisting of 1-androstendiol, 1-androstendione, bolandiol, bolasterone, boldenone, boldione, calusterone, clostebol, danazol, dehydrochlormethyltestosterone, desoxymethyltestosterone, drostanolone, ethylestrenol, fluoxymesterone, formebolone, furazabol, gestrinone, 4-hydroxytestosterone, mestanolone, mesterolone, metenolone, methandienone, methandriol, methasterone, methyldienolone, methyl-1-testosterone, methylnortestosterone, methyltrienolone, methyltestosterone, mibolerone, nandrolone, 19-norandrostenedione, norboletone, norclostebol, norethandrolone, oxabolone, oxandrolone, oxymesterone, oxymetholone, prostanozol, quinbolone, stanozolol, stenbolone, 1-testosterone, tetrahydrogestrinone and trenbolone. Endogenous Anabolic Androgenic Steroids (AAS), such as selected from the group consisting of androstenediol, androstenedione, dihydrotestosterone, prasterone and testosterone.

5)

Other Anabolic Agents, such as selected from the group consisting of clenbuterol, selective androgen receptor modulators (SARMs), tibolone, zeranol and zilpaterol.

6)

Short-acting 82 agonists (SABAs), such as selected from the group consisting of bitolterol—Tornalate, fenoterol—Berotec, isoprenaline (INN) or isoproterenol (USAN)—Isuprel, levosalbutamol (INN) or levalbuterol (USAN)—Xopenex, orciprenaline (INN) or metaproterenol (USAN)—Alupent, pirbuterol—Maxair, procaterol, ritodrine—Yutopar, salbutamol (INN) or albuterol (USAN)—Ventolin, terbutaline—Bricanyl and albuterol—Ventolin/Proventil.

7)

Aromatase inhibitors such as including, but not limited to aminoglutethimide, anastrozole, exemestane, formestane, letrozole and testolactone.

8)

Selective estrogen receptor modulators (SERMs) including, but not limited to raloxifene, tamoxifen and toremifene.

9)

Other anti-estrogenic substances, including but not limited to clomiphene, cyclofenil and fulvestrant.

10)

Diuretics, such as selected from the group consisting of acetazolamide, amiloride, bumetanide, canrenone, chlorthalidone, etacrynic acid, furosemide, indapamide, metolazone, spironolactone, thiazides, triamterene, epitestosterone and probenecid.

11)

Stimulants, such as selected from the group consisting of adrafinil, adrenaline, amfepramone, amiphenazole, amphetamine, amphetaminil, benzphetamine, benzylpiperazine, bromantan, cathine, clobenzorex, cocaine, cropropamide, crotetamide, cyclazodone, dimethylamphetamine, ephedrine, etamivan, etilamphetamine, etilefrine, famprofazone, fenbutrazate, fencamfamin, fencamine, fenetylline, fenfluramine, fenproporex, furfenorex, heptaminol, isometheptene, levmethamfetamine, meclofenoxate, mefenorex, mephentermine, mesocarb, methamphetamine (D−), methylenedioxyamphetamine, methylenedioxymethamphetamine, methylamphetamine, methylephedrine, methylphenidate, modafinil, nikethamide, norfenefrine, norfenfluramine, octopamine, ortetamine, oxilofrine, parahydroxyamphetamine, pemoline, pentetrazol, phendimetrazine, phenmetrazine, phenpromethamine, phentermine, 4-phenylpiracetam (carphedon), prolintane, propylhexedrine, selegiline, sibutramine, strychnine and tuaminoheptane.

12)

Antibiotics such as selected from the group consisting of Vancomycin, Teicoplanin, Linezolid, Daptomycin, Trimethoprim/sulfamethoxazole, Doxycycline, Ceftobiprole,

Ceftaroline, Clindamycin, Dalbavancin, Fusidic acid, Mupirocin (topical), Omadacycline, Oritavancin, Tedizolid, Telavancin, Tigecycline, Pseudomonas aeruginosa, Carbapenems, Ceftazidime, Cefepime, Ceftobiprole, Ceftolozane, Fluoroquinolones, Piperacillin, Ticarcillin, Streptogramins, Tigecycline, Daptomycin, Amikacin, Gentamicin, Kanamycin, Neomycin, Netilmicin,

Tobramycin, Paromomycin, Streptomycin, Spectinomycin, Ansamycins, Geldanamycin, Herbimycin, Rifaximin, Carbacephem, Loracarbef, Carbapenems, Ertapenem, Doripenem, Imipenem, Meropenem, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cephalosporins, Cefixime, Cefdinir, Cefditoren, Cefoperazone, Cefoperazone, Cefotaxime, Cefpodoxime, Ceftazidime, Ceftibuten, Ceftizoxime, Moxalactam, Ceftriaxone, Cephalosporins, Cefepime, Cephalosporins, Ceftaroline fosamil, Ceftobiprole, Glycopeptides, Teicoplanin, Vancomycin, Telavancin, Dalbavancin, Oritavancin, Lincosamides, Clindamycin, Lincomycin, Lipopeptide, Daptomycin, Macrolides(Bs), Azithromycin, Clarithromycin, Erythromycin, Roxithromycin, Telithromycin, Spiramycin, Fidaxomicin, Monobactams, Aztreonam, Nitrofurans, Furazolidone, Nitrofurantoin(Bs), Oxazolidinones(Bs), Posizolid, Radezolid, Torezolid, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, Ticarcillin, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole, Sulfonamidochrysoidine (archaic), Tetracyclines, Demeclocycline, Doxycycline, Metacycline, Minocycline, Oxytetracycline, Tetracycline, Drugs against mycobacteria, Clofazimine, Dapsone, Capreomycin, Cycloserine, Ethambutol(Bs), Ethionamide, Isoniazid, Pyrazinamide, Rifampicin, Rifabutin, Rifapentine, Streptomycin.

13)

Compounds selected from the group consisting of Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole and Trimethoprim.

Thus the invention can find use in the detection/quanitification of many different groups of compounds/small molecules.

The binding affinity to the analyte binding protein (3) may be different for the analyte and the analyte analogue. Thus, in an embodiment said analyte analogue (6) has a higher, a lower or an equal affinity for the binding site (4) in the analyte binding protein (3), compared to the analyte (2), preferably the binding affinity is lower.

In a further embodiment, said analyte binding protein (3) is selected from the group consisting of an antibody or fragment thereof, such as a Fab fragment, a

F(ab′)2, a Fv, a Fd, a dAb, a scFv fragment or a single-domain antibody (sdAb) such as a Nanobody, and an affibody.

In yet an embodiment, the analyte binding protein is selected from the group consisting of idarucizumab, an anti-apixaban antibody and an anti-linezolid antibody.

Preferably, the optical signal pairs are fluorescent pairs. Different types of fluorescent pairs may find use as optical signal pairs in the method of the invention. Thus in an embodiment the fluorescent pair (5) is selected from the group consisting of fluorescent pairs.
I. A FRET pair such as Cy3 and Cy5, Cy5.5 and Cy7, Atto425 and Atto 520, Atto488 and Atto590, Atto488 and Atto647N, Atto488 and Atto550, Atto488 and Atto565, Atto488 and Atto655, Atto550 and Atto 647N, Atto323 and Atto647N, Atto532 and Atto655, Atto550 and Atto655, Atto550 and Atto590, Atto550 and Atto647N, Atto565 and Atto590, Atto565 and Atto647N, Atto590 and Atto655, Atto590 and Atto620, Atto590 and Atto647N, Atto590 and Atto680, Atto620 and Atto680, Alexa Fluor 350 and Alexa Fluor 488, Alexa Fluor 488 and Alexa Fluor 546, Alexa Fluor 488 and Alexa Fluor 555, Alexa Fluor 488 and Alexa Fluor 568, Alexa Fluor 488 and Alexa Fluor 594, Alexa Fluor 488 and Alexa Fluor 647, Alexa Fluor 546 and Alexa Fluor 568, Alexa Fluor 546 and Alexa Fluor 594, Alexa Fluor 546 and Alexa Fluor 647, Alexa Fluor 555 and Alexa Fluor 594, Alexa Fluor 555 and Alexa Fluor 647, Alexa Fluor 568 and Alexa Fluor 647, Alexa Fluor 594 and Alexa Fluor 647, TMR and Texas Red, Texas Red and Cy5, FAM and Cy5, FAM and TAMRA, preferably the FRET pair is Cy3 and Cy5;
II. a fluorophore-quencher pair such as Alexa Fluor 350 and QSY 35, Alexa Fluor 350 and dabcyl, Alexa Fluor 350 and BHQ-0, Alexa Fluor 350 and TQ1, Alexa Fluor 350 and TQ2, Alexa Flour 350 and BHQ-1, Alexa Fluor 488 and QSY 35, Alexa Fluor 488 and dabcyl, Alexa Fluor 488 and QSY 7&9, Alexa Fluor 488 and BHQ-0, Alexa Fluor 488 and BHQ-1, Alexa Fluor 488 and TQ1, Alexa Fluor 488 and TQ2, Alexa Fluor 488 and TQ3, Alexa Fluor 546 and QSY 35, Alexa Fluor 546 and dabcyl, Alexa Fluor 546 and QSY 7&9, Alexa Fluor 546 and BHQ-2, Alexa Fluor 546 and BHQ-1, Alexa Fluor 546 and BHQ-3, Alexa Fluor 546 and TQ2, Alexa Fluor 546 and TQ3, Alexa Fluor 546 and TQ4, Alexa Fluor 546 and QSY 21, Alexa Fluor 555 and QSY 7&9, Alexa Fluor 555 and BHQ-2, Alexa Fluor 555 and BHQ-1, Alexa Fluor 555 and QSY 21, Alexa Fluor 555 and BHQ-3, Alexa Fluor 555 and TQ2, Alexa Fluor 555 and TQ3, Alexa Fluor 555 and TQ4, Alexa Fluor 568 and QSY 7&9, Alexa Fluor 568 and BHQ-2, Alexa Fluor 568 and QSY 21, Alexa Fluor 594 and QSY 21, Alexa Fluor 594 and QSY 7&9, Alexa Fluor 594 and BHQ-2, Alexa Fluor 594 and BHQ-3, Alexa Fluor 594 and TQ3, Alexa Fluor 594 and TQ4, Alexa Fluor 594 and TQS, Alexa Fluor 647 and QSY 21, Alexa Fluor 647 and BHQ-3, Alexa Fluor 647 and TQ4, Alexa Fluor 647 and TQS, Alexa Fluor 647 and TQ6, Alexa Fluor 680 and TQS, Alexa Fluor 680 and TQ6, Alexa Fluor 680 and TQ7, Alexa Fluor 750 and TQ6, Alexa Fluor 750 and TQ7, Cy3 and BHQ-1, Cy3 and BHQ-2, Cy3 and BHQ-3, Cy3 and QSY 7&9, Cy3 and QSY 21, Cy3 and TQ2, Cy3 and TQ3, Cy3 and TQ4, Cy3 and Eclipse Dark Quencher, Cy3.5 and BHQ-2, Cy3.5 and Deep Dark Quencher II, Cy5 and BHQ-3, Cy5 and QSY 21, Cy5 and BHQ-2, Cy5 and BHQ-1, Cy5 and TQ4, Cy5 and TQ5, Cy5 and TQ6, Cy5 and Deep Dark Quencher II, Cy5.5 and BHQ-3, Cy5.5 and QSY 21, Cy5.5 and TQ5, Cy5.5 and TQ6, Cy5.5 and TQ7, Cy7 and TQ6, Cy7 and TQ7, TAMRA and BHQ-1, TAMRA and BHQ-2, TAMRA and QSY 7&9, TAMRA and dabcyl, FAM and BHQ-1, FAM and BHQ-2, FAM and BHQ-0, FAM and QSY 7&9, FAM and dabcyl, FAM and TQ1, FAM and TQ2, FAM and TQ3, Texas Red and BHQ-2, Texas Red and BHQ-1, Texas Red and BHQ-3, Texas Red and QSY 7&9, Texas Red and QSY 21, Texas Red and TQ3, Texas Red and TQ4, Texas Red and TQ5, Texas Red and dabcyl, TET and BHQ-1, TET and BHQ-2, ATT0488 and BHQ-0, ATT0532 and BHQ-1, ATT0532 and Eclipse Dark Quencher, ATT0550 and BHQ-2, ATT0550 and BHQ-1, ATT0550 and BHQ-3, ATT0550 and TQ2, ATT0550 and TQ3, ATT0550 and TQ4, ATT0550 and QSY 7&9, ATT0550 and QSY 21, ATT0550 and Deep Dark Quencher II, ATT0565 and BHQ-2, ATT0565 and BHQ-1, ATT0565 and BHQ-3, ATT0565 and TQ2, ATT0565 and TQ3, ATT0565 and TQ4, ATT0565 and QSY 7&9, ATT0565 and QSY 21, ATT0565 and Deep Dark Quencher II, ATT0590 and BHQ-2, ATT0590 and Deep Dark Quencher II, ATT0620 and BHQ-2, ATT0620 and Deep Dark Quencher II, ATT0620 and BHQ-3, HEX and TQ2, HEX and TQ3, HEX and TQ4, HEX and BHQ-1, HEX and BHQ-2, HEX and BHQ-3, HEX and QSY 7&9, HEX and QSY 21, TET and TQ2, TET and TQ3, TET and TQ4, TET and BHQ-1, TET and BHQ-2, TET and BHQ-3, TET and QSY 7&9, TET and QSY 21, JOE and TQ2, JOE and TQ3, JOE and TQ4, JOE and BHQ-1, JOE and BHQ-2, JOE and BHQ-3, JOE and QSY 7&9, JOE and QSY 21.

In an embodiment, the fluorescent pairs are Quantum dots.

In another embodiment, the first member (5A) of the optical signal pair (5) is (covalently) coupled to the analyte binding protein (3) through a first linker, preferably an oligonucleotide linker, even more preferably a DNA linker. In yet another embodiment, the second member (5B) of the optical signal pair (5) is (covalently) coupled to the analyte analogue (6) through a second linker, preferably an oligonucleotide linker, even more preferably a DNA linker. Preferably, the members of the optical pairs are covalently coupled, even more preferably through DNA linkers.

Advantages of using oligonucleotide linkers are:
Increased solubility. The oligonucleotide-protein conjugate is more soluble in aqueous fluids such as buffers, plasma or blood than the protein alone. This is of importance when the protein—oligonucleotide conjugate is dissolved by the plasma in the method of the invention, such as the solid support assay.
Ease of purification for analyte analogue and analyte binding protein. It is possible to produce pure protein (antibody, Fab)-oligonucleotide-dye conjugates, since they can be separated from impurities of non-labelled protein or protein with more labels by gel electrophoresis or ion exchange chromatography.
Easy covalent coupling to the analyte binding protein (3).
Adjustable affinity between the oligonucleotide/DNA strands. Thus, it is possible through design of the oligonucleotide linkers to control the amount of hybridization and thereby fine-tune the associated FRET signal of the assay.

In the event oligonucleotides are used as linkers, it may be an advantage that the oligonucleotides do not hybridize to each other. Thus, in an embodiment, the first oligonucleotide linker and the second oligonucleotide linker are non-complementary. In a specific embodiment, the first oligonucleotide linker and the second oligonucleotide linker are identical in sequence.

In an embodiment, the first linker and the second linkers consist only of an oligonucleotide, preferably a DNA linker. In another embodiment, the first linker and the second linker does not comprise any peptides, proteins, alkyls or polyethylene glycol (PEG).

In an embodiment, the first and/or second oligonucleotide linker comprises one or more modified/artificial nucleotides, such as LNA, PNA, L or D acyclic threoniniol nucleic acid (aTNA), 2-fluoro-DNA or 2-MeO-DNA, and morpholino-DNA.

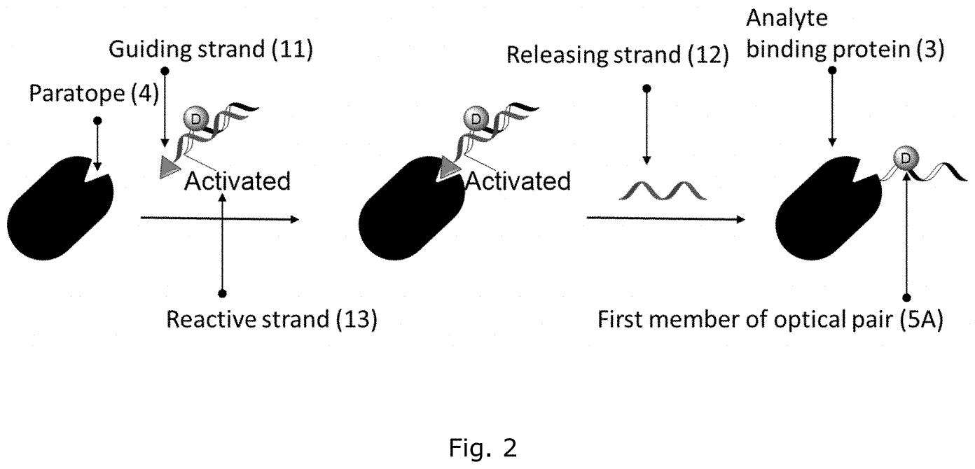

In yet an embodiment the DNA strands used in the invention are SEQ ID NO's 1 and 4. With reference to FIGS. 1 and 2, SEQ ID NO: 1 is the reactive strand (13) comprising a Cy3, SEQ ID NO: 2 is the guiding strand (11) assisting in the coupling of SEQ ID NO: 1 to the analyte binding protein, SEQ ID NO: 3 is the releasing strand (12) assisting in removal of the guiding strand after coupling, and SEQ ID NO: 4 is the sequence coupled to the analyte analogue comprising a Cy5. It is to be understood that Cy3 and Cy5 may be substituted with other fluorescent pairs according to the invention. The skilled person could also develop other DNA strands, which may be functional in the assay of the invention. For further details, see also example 3.

In a further embodiment, the first oligonucleotide and the second oligonucleotide are partly complementary, such as by having maximum 10 complementary bases such as maximum 9 or such as maximum 8 complementary bases. By having a weak complementary between the first oligonucleotide and the second oligonucleotide, binding efficiency can be increased for weak bindings between analyte analogue (6) and the analyte binding protein (3) or for increasing the FRET signal. The skilled person knows of methods for determining complementary (sequence identify), such as by using the software NUPACK (http://www.nupack.org/), or other oligonucleotide tools available online.

In an embodiment, the first oligonucleotide is at most 90% complementary to the second oligonucleotide, such as at most 80%, such as at most 70%, such as at most 60%, such as at most 50%, such as at most 40%, preferably at most 30%, more preferably at most 20%, most preferably at most 10% complementary to the second oligonucleotide. Preferably, the first and second oligonucleotides are DNA.

In the present context, when referring to “complementary”, G pairs to C, A pairs to T and U and vice versa. In some embodiments, G may also pair to U and vice versa to form a so-called wobble base pair. A wobble base pair is a non-Watson-Crick base pairing between two nucleotides in RNA molecules. The four main wobble base pairs are guanine-uracil, inosine-uracil, inosine-adenine, and inosine-cytosine (G-U, I-U, I-A and I-C).

Thus, in the present context “complementarity” is a measure of complementarity between nucleic acids at nucleotide level. The nucleic acid complementarity may be determined by comparing the nucleotide sequence in a given position in each sequence of the first and second linkers when the sequences are aligned.

To determine the percent identity of two nucleic acids sequences, the sequences are aligned for optimal comparison purposes (e.g., gaps may be introduced in the sequence of a first nucleic acid sequence for optimal alignment with a second nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical nucleotides. Alternatively, alignment of two sequences for the determination of percent identity may be accomplished using a mathematical algorithm. Such an algorithm is incorporated into the NBLAST program of (Altschul et al. 1990). BLAST nucleotide searches may be performed with the NBLAST program, score=100, wordlength=12, to obtain nucleotide sequences homologous to a nucleic acid molecules of the invention.

To obtain gapped alignments for comparison purposes, Gapped BLAST may be utilized. Alternatively, PSI-Blast may be used to perform an iterated search, which detects distant relationships between molecules. When utilising the NBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

In yet another embodiment, the first member (5A) of the fluorescent pair (5) is covalently coupled to the analyte binding protein (3) through a first linker. In yet another embodiment the second member (5B) of the fluorescent pair (5) is covalently coupled to the analyte analogue (3) through a second linker. In yet an embodiment, the first linker is not a peptide or a protein-based molecule having affinity for the analyte binding protein (3), such as an antibody or fragment thereof.

The magnitude of the FRET signal depends on the proximity between the first member (5A) and second member (5B) of the optical signal pair (5). Thus, through adjustment of the length of the linkers, the FRET signal can be fine-tuned by altering the proximity of the first member (5A) and second member (5B) of the optical signal pair (5). Therefore, in an embodiment, the first linker is an oligonucleotide consisting of between 2 and 100 nucleotides, such as between 2 and 75 nucleotides, preferably between 5 and 40 nucleotides. In another embodiment, the second linker is an oligonucleotide consisting of between 2 and 100 nucleotides, such as between 2 and 75 nucleotides, preferably between 5 and 40 nucleotides. In a further embodiment, the first linker and second linker are oligonucleotides linkers, each comprising at least 5 nucleotides, preferably at least 10 nucleotides. In yet another embodiment, the first linker and second linker are oligonucleotides linkers, each comprising at most 100 nucleotides, such as at most 75 nucleotides, preferably at most 50 nucleotides. Preferably, the oligonucleotides are DNA.

In an embodiment, the first member (5A) of the optical signal pair (5) is a donor fluorophore of a FRET pair and the second member (5B) of the optical signal pair (5) is an acceptor fluorophore of a FRET pair. In another embodiment, the first member (5A) of the optical signal pair (5) is an acceptor fluorophore of a FRET pair and the second member (5B) of the optical signal pair (5) is a donor fluorophore of a FRET pair. Preferably, the donor and acceptor fluorophores are coupled to the first and second oligonucleotide linkers, preferably DNA linkers.

Different types of references signals may be used in a method according to the present invention. Thus, in an embodiment the reference fluorescent signal is a negative control (such as a corresponding sample known not to comprise the analyte in question).

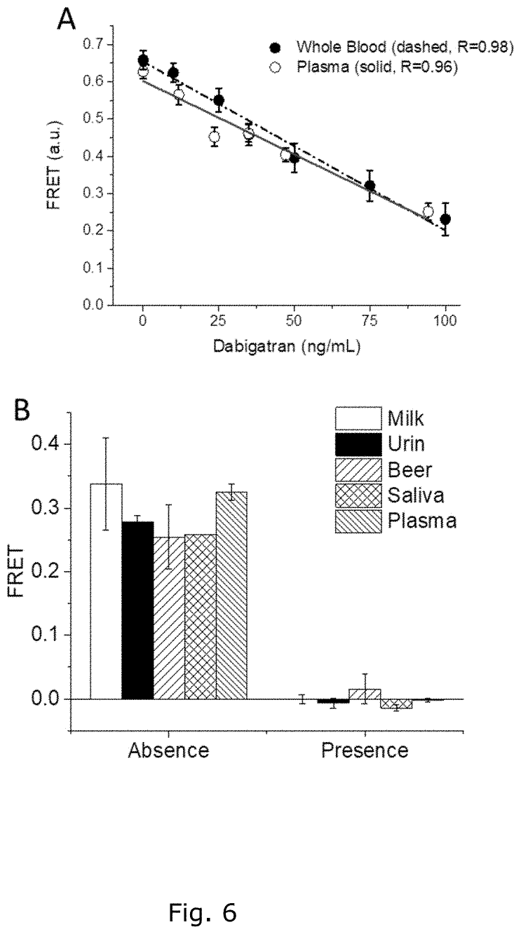

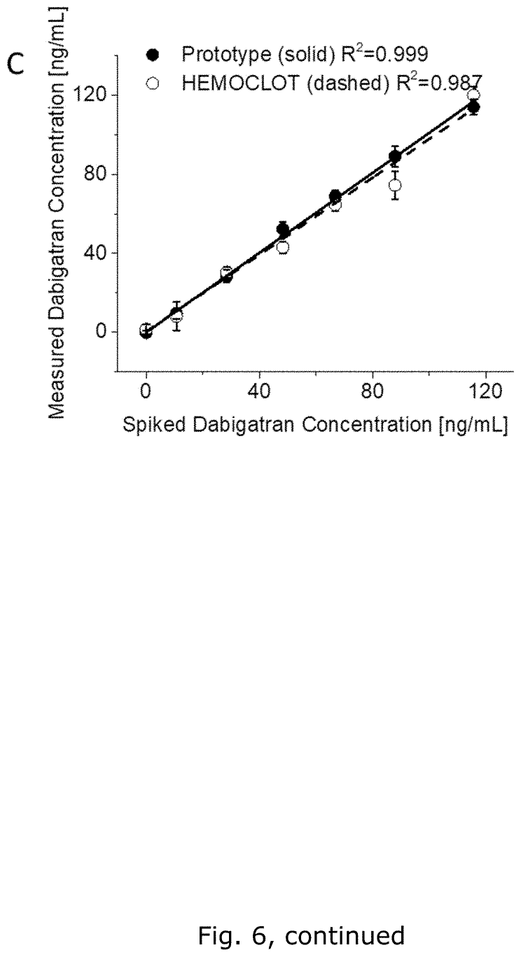

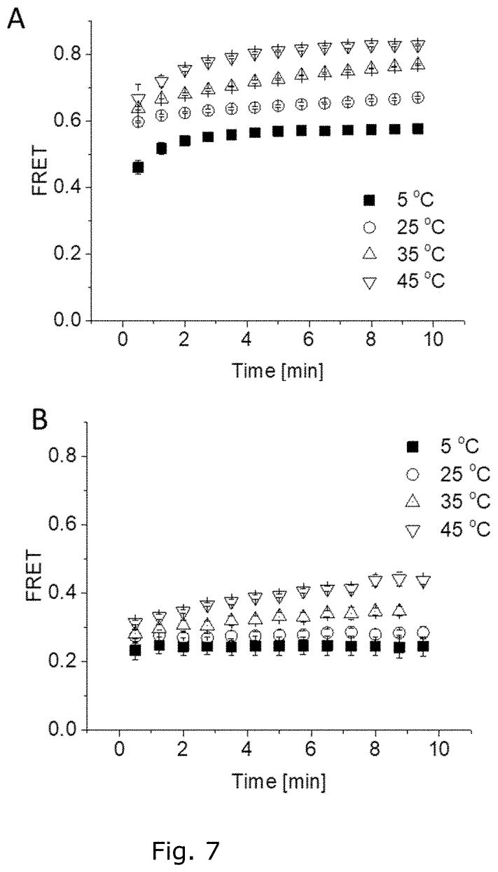

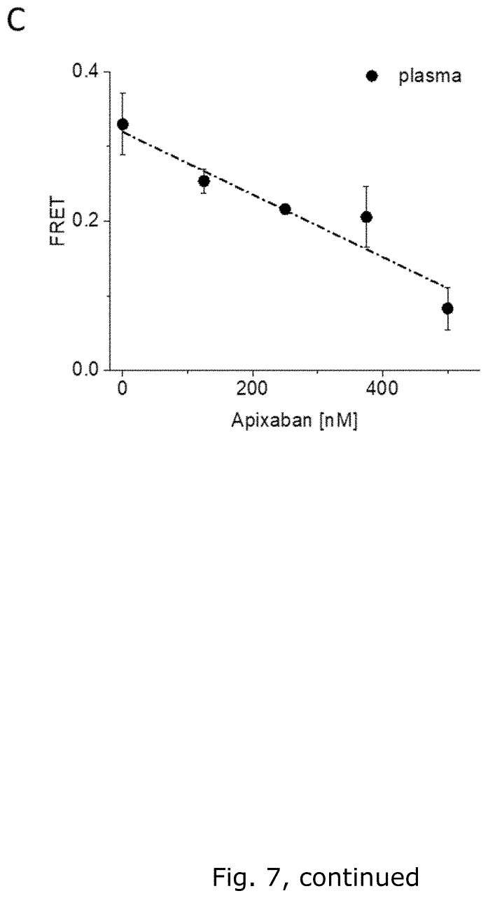

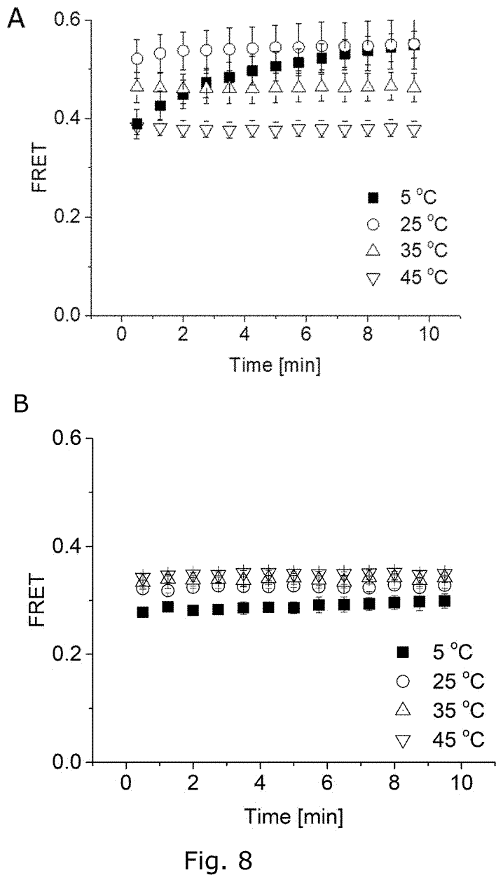

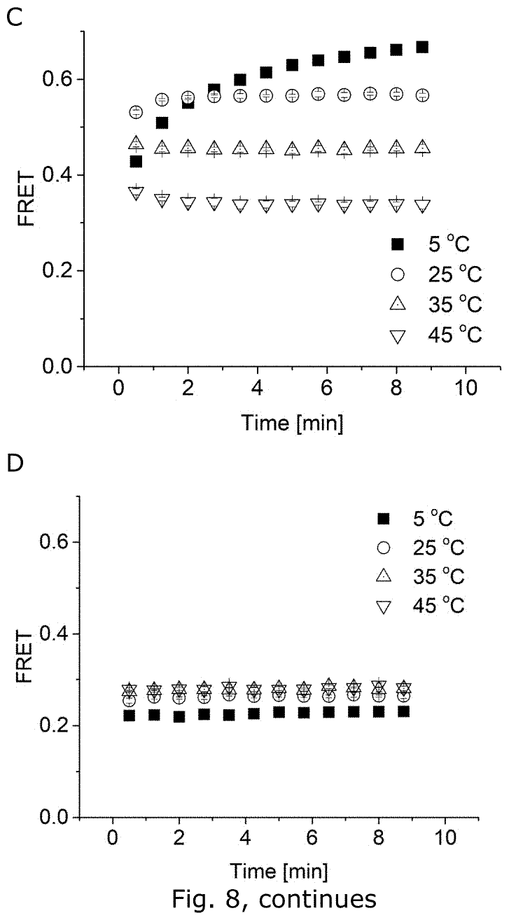

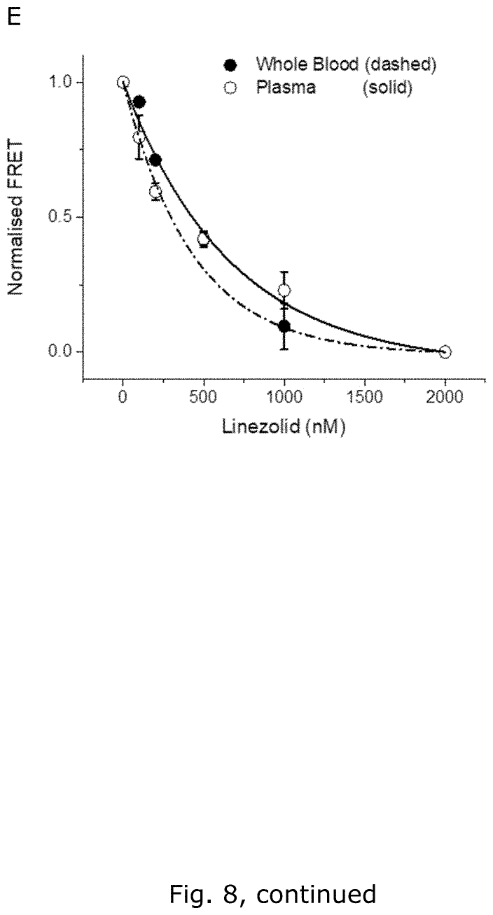

When the concentration of the analyte is determined, it may be determined within a certain range. Thus, in an embodiment, the concentration determination is in the range 0 nM to 2000 nM, such as in the range 0 to 1500 nM, such as 0.1 to 1200 nM, such as in the range 0.1-200 nM, such as in the range 2-20 nM OR such as in the range 10 to 1000 nM, such as 10 to 400 nM, or such as 10 to 200 nM. In examples 3-8 concentrations in the range 0-100 nM (FIG. 6A), 0-120 nM (FIG. 6C), 0-500 nM (FIG. 7C), 0-1000 nM (FIG. 1C), and 0-2000 nM (FIG. 8E) are determined.

The method of the invention may take place in solution and/or using a solid support format. Thus, in an embodiment, said analyte binding protein (3) and said analyte analogue (6) are provided on a (porous) solid or (porous) semi-solid support.

In yet an embodiment, said analyte binding protein (3) and said analyte analogue (6) are provided on independently distinct regions on (porous) the semi-solid or (porous) solid support. Examples 4, 6 and 8 show data for the method in a solid support format. In a further embodiment, the semi-solid or solid support is selected from the group consisting of a paper, a membrane, a polymeric gel, a fiber, a polymer, a polymeric fiber, a polymeric particle, a polymeric microparticle or an array.

The order of which the components are brought in contact with each other can also vary. Thus, in an embodiment said sample (2) is brought in contact with the analyte binding protein (3) before being brought in contact with said analyte analogue (6). In yet another embodiment, said analyte analogue (6) is brought in contact with a mixture of the sample (2) and the analyte binding protein (3). Example 11 documents that the order of mixing the assay species influences on the efficiency of the assay.

In yet another embodiment, said sample is provided in a dissolved state to the first region and is subsequently transported to the second region and subsequently detected, e.g. in a dedicated detection chamber (9). In a related embodiment, said transport is by capillary forces.

It is important for FRET analysis (or for fluorophore-quencher pairs) that the molecules are in close proximity. Thus, in an embodiment, the distance between the first member 5A and the second member 5B, when the analyte analogue is bound to the analyte binding protein, is in the range 1-100 Angstrom, such as 10-100 Ångstrom, such as 10-50 Ångstrom.

In another embodiment, the distance between the first member (5A) and the second member (5B), when the analyte analogue is bound to the analyte binding protein, is in the range 10-30 Ångstrom, such as 10-20 Ångstrom, such as 10-15 Ångstrom.

The proximity between the first member (5A) and the second member (5B) is amongst others influenced by the coupling technique of the first member (5A) to the analyte binding protein (3). Thus, coupling techniques that ensure coupling of the first member (5A) near the binding site (4) for the analyte (1) is preferred. In an embodiment, the first member (5A) is coupled to the analyte binding protein (3) using a first guiding oligonucleotide (DNA) strand (11) conjugated to an analyte (1), and hybridized to a reactive oligonucleotide (DNA) strand (13) conjugated to the first member (5A), and a reactive group. In another embodiment, the reactive group binds in the vicinity of the binding site (4) for the analyte (1), such as within 5-30 Å of the binding site (4). In a further embodiment, the guiding strand (11) binds non-covalently to the binding site (4). In yet another embodiment, a releasing strand (12) is applied to remove the guiding strand (11) by strand displacement. The analyte binding protein (3) may be e.g. natural and recombinant proteins, such as antibodies Fab domains and other proteins with affinity for a small molecule ligand.

Alternative coupling techniques can be employed as well with the method described herein. Thus, in an embodiment, the analyte binding protein (3) is an antibody and the first member (5A) is coupled to the Fab region of said antibody. For coupling to the Fab region, the first member (5A) may be conjugated to a linker, such as an oligonucleotide linker, preferably a DNA linker. In another embodiment, the first member (5A) is coupled to the analyte binding protein (3) by thiol coupling, such as coupling to cysteine residues of the analyte binding protein (3). For thiol coupling to the analyte binding protein (3), the first member (5A) may be conjugated to a linker, such as an oligonucleotide linker, preferably a DNA linker.

Optical detection signals can be determined using different setups. Thus, in an embodiment, the optical signal is determined using spectroscopy, such as FRET as shown in FIG. 1B and FIG. 1C.

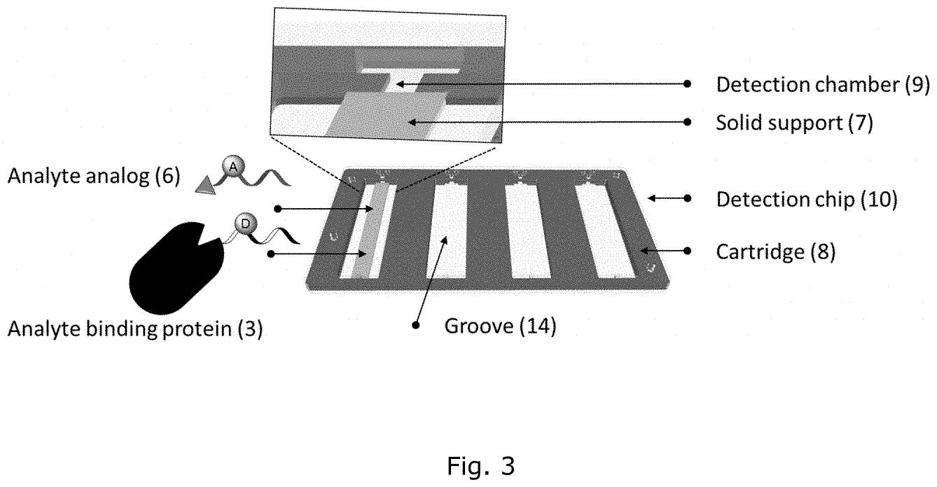

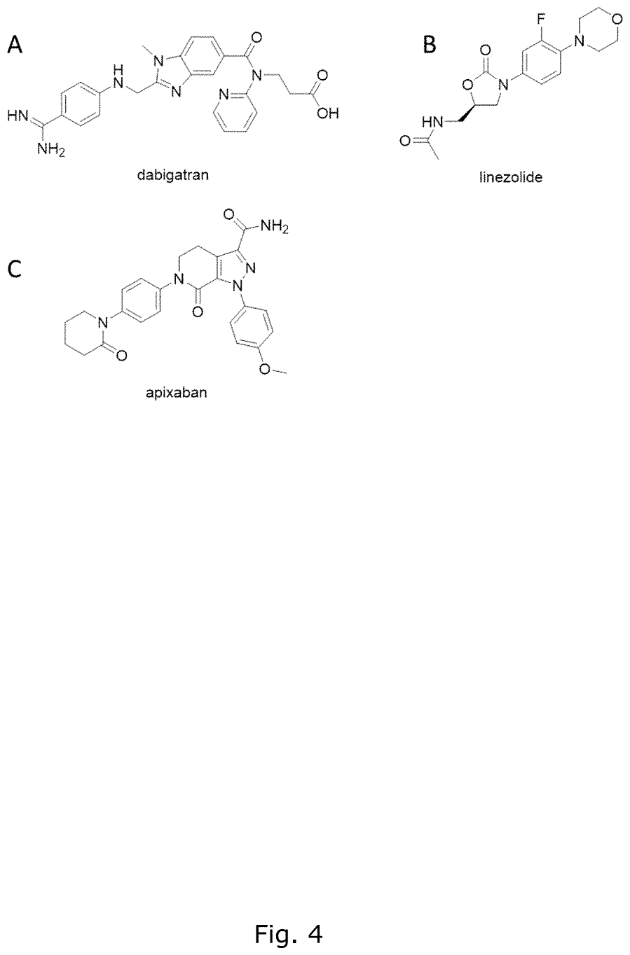

It is to be understood that the method of the invention may be performed in a detection chip (10) according to the invention using a solid support (7) and a cartridge (8) according to the invention as further described below. See also FIG. 3.

Kit

The present invention also relates to a kit enabling performing the method of the invention. Thus, in an aspect the invention relates to a kit comprising
I. a first vial comprising an analyte binding protein (3), said analyte binding protein comprising
   a binding site (4) for an analyte (1); and
   a covalently linked first member (5A) of an optical signal pair (5);
II. a second vial comprising an analyte analogue (6), said analyte analogue (6) being covalently coupled to a second member (5B) of the optical signal pair (5);
III. optionally, a porous solid support material (7) for depositing the content of the first vial and the second vial at different (indicated) distinct regions; and
IV. optionally, a cartridge (8) for mounting the solid support material (7).

As shown in examples 3, 5 and 7 the method of the invention may be performed in a setup using the kit components of the invention.

Solid Support

In yet an aspect the invention relates to a (porous) solid support which may be used in the method of the invention or form part of a kit according to the invention, where component I. and component II. of the kit are deposited on the solid support. Thus, an aspect of the invention relates to a porous solid support (7) comprising
a) a first region comprising an analyte binding protein (3), said analyte binding protein (3) comprising
   a binding site (4) for an analyte (1); and
   a covalently linked first member (5A) of an optical signal pair (5);
b) a second region comprising an analyte analogue (6), said analyte analogue (6) being covalently coupled to a second member (58) of the optical signal pair (5).

In a variant of the solid support, the solid support is configured for receiving a sample, and bringing said sample in contact with the analyte binding protein (3) before bringing said sample in contact with said analyte analogue (6). Thus, in an embodiment, the first region and the second region are arranged in serial connection on the solid support. In another embodiment, the solid support comprises an anterior end and a posterior end, wherein the anterior end comprises the first region and the posterior end comprises the second region. In a further embodiment, the solid support comprises an inlet for receiving a sample. In another embodiment, the inlet is positioned in the anterior region of the solid support. In an embodiment, the posterior end of the solid support is placed in fluid connection with a detection chamber (9). In a further embodiment, the posterior end of the solid support or the detection chamber (9) comprises an outlet. Preferably, the sample moves through the solid support by capillary forces. As shown in example 11, the order of mixing may improve the efficiency of the assay. Thus, in a preferred embodiment, the solid support is configured for receiving a sample, and bringing said sample in contact with the analyte binding protein (3) before bringing said sample in contact with said analyte analogue (6).

In a preferred embodiment, said first region and said second region are at independent regions at the solid support (7). Such as setup is depicted in FIG. 3. In yet an embodiment, the solid support (7) is in a dry state. The solid support may consist of or comprise different materials. Thus, in an embodiment, said solid support comprises or consist of a fiber, including glass fibers and in particular Fusion 5; paper, a membrane, a polymeric gel, a polymer, a polymeric fiber, a polymeric particle, a polymeric microparticle or an array. An advantage of using such as porous solid support (preferably glass fibers) is that e.g. a whole blood sample will separate into blood cells and blood plasma, with blood plasma being able to be transported through the solid support. Furthermore, the glass fibers are optimized for both storage of reagents as well as rapid release of the aforementioned reagents.

Cartridge

The porous solid support (7) of the invention may be mounted in a cartridge (8). Thus, in yet an embodiment, the solid support is mounted in a cartridge (8). In another embodiment, the cartridge (8) further comprises a detection chamber (9) or is in fluid connection to a detection chamber (9). In an additional embodiment, said detection chamber (9), is adapted to receive fluid (to be investigated for the presence, absence and/or concentration of the analyte) when said fluid has passed through said first region and said second region of the porous solid support (mounted in the cartridge (8)). In yet an embodiment, the porous solid support is adapted to transport a fluid by the use of capillary forces from, such as through the first region, to the second region and to the detection chamber (9). In a further embodiment, the cartridge (8) comprises an outlet. In another embodiment, the outlet is positioned in the detection chamber (9). Again, preferably, the sample moves through the solid support by capillary forces.

In an embodiment the cartridge (8) comprises or consists of a thermoplastic, preferably poly(methyl methacrylate) (PMMA). PMMA also known as acrylic or acrylic glass as well as by the trade names Crylux, Plexiglas, Acrylite, Lucite, and Perspex among several others, is a thermoplastic (which may be transparent) often used in sheet form as a lightweight or shatter-resistant alternative to glass.

In an embodiment, the cartridge (8) is designed to enable the fluid (sample (2)) to migrate by capillary forces from/through the solid support (7) to the detection chamber (9) and stop when the detection chamber (9) is filled.

In another embodiment, the part of the cartridge (8) comprising the detection chamber (9) comprises an optically transparent material, such as optically transparent PMMA, allowing for optical determination of the analyte in the sample. In yet an embodiment the remaining part of the cartridge (8) is optically non-transparent, such as optically non-transparent PMMA.

In a further embodiment, the cartridge (8) is adapted to be mounted with several solid supports (7) each solid support being in connection with individual detection chambers (9). Such a setup is shown in FIG. 3.

Thus, an aspect of the invention relates to a cartridge (8) mounted with the porous solid support (7) according to the invention.

In yet an embodiment, the invention relates to a cartridge (8) comprising a groove (14) for mounting of the solid support (7) and a detection chamber (9), wherein the detection chamber (9) is made of an optically transparent material allowing for optical determination of the analyte (1) in the sample (2). In FIG. 3, an example is provided where the cartridge comprises four grooves (14), with a solid support mounted in the left groove. The skilled person would also understand the term “groove” as a “depression” or “chamber” in the cartridge. The cartridge may comprise one or more grooves (14) for mounting one or more solid supports. In an embodiment, the cartridge comprises 1-50 grooves, 1-30, such as 1-5, or such as 1-4, 1-3, 1-2, or 1 groove, alternatively 5-50 grooves, such as 10-40 or 20-30 grooves. Each groove of course has to be in fluid connection with an independent detection chamber (9) as also apparent from FIG. 3.

Detection Chip

The invention relates to a detection chip (10). Thus, in an aspect the invention relates to a detection chip (10) comprising the solid support (7) according to the invention mounted in a cartridge (8) according to the invention. Phrased in another way, the detection chip (10) is adapted for the fluid (such as a blood sample (2)) to be applied to one end of the solid support (7) loaded in the cartridge (8) (in sum the detection chip (10)). The fluid may move by capillary forces through the first region and afterwards through the second region of the solid support (7) and finally end up in the detection chamber (9), where the presence, absence or concentration of an analyte (1) in the sample (2) may be determined. See also FIG. 3.

Uses

In yet another aspect, the invention relates to the use of the kit according to the invention and/or the porous solid support (7) according to the invention and/or the cartridge (8) according to the invention and/or the detection chip (10) for determining the presence, absence and/or concentration of an analyte (1) in a sample (2).

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1—Method of the Invention

The analyte binding protein (3) being covalently linked to the first member (5A) of a fluorescent pair (5), applied for the assay may be prepared in the following way. As shown in FIG. 2, a first guiding DNA strand (11) is conjugated to a small molecule analyte (1), and hybridized to a reactive strand (13) conjugated to a dye (5A), and a reactive group. The guiding strand (11) binds non-covalently to the paratope (4) of the protein (coming analyte binding protein (3)) and directs, via the duplex, the reactive group to bind in the vicinity of the binding site (4) for the analyte (1). Finally, a releasing strand (12) is applied to remove the guiding strand (11) by strand displacement. By this method, it is possible to produce an analyte binding protein (3) containing a dye (5A) close to the paratope (4) in high yields on natural and recombinant proteins (such as antibodies). In here this have been shown for antibodies, Fab domains and other proteins with affinity for a small molecule ligand. It is considered a generic technology that can be applied for proteins that bind to a molecule target.

The assay in action using the above described produced analyte binding protein (3) is shown in FIG. 1A for an antibody Fab domain. All conjugates are prepared and purified. The assay is based on inhibitory binding of the analyte (1) to the analyte binding protein (3), the analyte binding protein (3) being covalently linked to the first member (5A) of a fluorescent pair (5). This inhibitory binding of the analyte (1) to the analyte binding protein (3) has consequences for the binding of the subsequently added analyte analogue (6) covalently coupled to a second member (5B) of the fluorescent pair (5).

In the presence of the analyte (1), the paratope (4) for the analyte (1) of the analyte binding protein (3) is blocked and when the analyte binding protein (3) is exposed to the analyte analogue (6), the analyte analogue (6) cannot bind to the protein (3) (Shown in the top sequence for presence of analyte). This results in emission only from the FRET donor (5A or 5B) when the donor is excited and serves as a clear zero point signal.

In the absence of the analyte (1) the analyte analogue (6) can bind to the analyte binding protein (3). Hence, the proximity of the first member (5A) and the second member (5B) of the fluorescent pair (5) efficiently provide a FRET signal, i.e. when the donor of the fluorescent pair is excited, energy is transferred to the acceptor and emission from the acceptor is observed. This serves as the maximal signal. If the max signal is used as a reference, any sub-stoichiometric amount of the analyte (relative to antibody) can be quantified based on a calibration curve. The readout using a fluorometer for dabigatran at different stoichiometry relative to antibody is shown in FIG. 18. Examples 3-8 show detection of different analytes using this method.

### Example 2—Solid Support Format

Explanatory example of one embodiment of the method of the invention, wherein the method is performed on a solid support with reference to the figures.

The solid support assay is conducted on a conjugate pad technology in conjunction with liquid phase fluorescence spectroscopy using a transparent detection chamber (9). The conjugate pad (e.g. pieces of porous paper, microstructured polymer or sintered polymer) is prepared as shown in FIG. 3.

The analyte binding protein (3) (being covalently linked to the first member (5A) of a fluorescent pair (5)) is spotted on one position of a solid support (7) while the analyte analogue (6) (being covalently coupled to a second member (58) of the fluorescent pair (5)) is spotted on another point (further to the left relative to the first spotting on FIG. 3) of the same solid support. When the sample (2) (e.g. plasma or other complex matrix to be tested for the presence and/or concentration of the analyte (1)) is applied at the opposite end of the detection chamber (9) to the solid support (7), wetting of the conjugate pad drives the solution through the two spots where the dried analyte binding protein (3) is first solvated and transported by the solution and secondly the analyte analogue (6) is solvated and transported. Thus, in one combined transport action the analyte binding protein (3) and analyte analogue (6) mix with the test-sample (comprising the analyte (1)) while flowing through the conjugate pad. Once the conjugate pad is soaked, the fluid migrates by capillary forces into the transparent detection chamber (9). The combined process, from applying the liquid sample to filling the transparent detection chamber (9), proceeds in less than 5 minutes.

Examples 4, 6 and 8 show detection of analytes in such a solid support format.

### Example 3—Dabigatran Competition Assay

Aim

The aim of these experiments was to detect the presence/concentration of the anticoagulant dabigatran in both buffer and plasma by measuring FRET using a spectrofluorometer.

Materials and Methods

An analyte binding protein (3) and an analyte analogue (6) is used for this experiment. The DNA-strands are modified with internal Cy3 or Cy5 fluorophores and contain 3′-amino modifier for the functionalization with dabigatran.

Strand Sequences:

| SEQ | | |
|---|---|---|
| ID | Name | Sequence |
| 1 | Reactive strand | 5′ ACATACAGCCTCG |
| | (13) | CATGAG-Cy3-CCC-Y 3′ |
| 2 | Guiding strand | 5′ X-GGGTCTCATGCG |
| | (11) | AGGCTTACGAAC 3′ |
| 3 | Release strand | 5′ GTTCGTAAGCCTCGC |
| | (12) | ATGAGACCCGTAGGCAAT |
| | | CCTAC 3′ |
| 4 | Analyte | 5′ TTTCACACTTCCTTA |
| | analogue (6) | CTGAGTCTATCTATTC- |
| | | Cy5-ACC- |
| | | Y 3′ |

X: 5′ Amino modifier C6 (from IDT DNA)
Y: 3′ Amino modifier (from IDT DNA)

The strands were purchased from Integrated DNA technologies (IDT DNA). In general, the chemicals were purchased from Sigma-Aldrich. Dabigatran was purchased at Cayman Chemical Company. Idarucizumab (Praxbind®) was supplied by Aarhus University Hospital.

Methods:

Modification of DNA Strands:

Preparation of the Reactive Strand (13):

The protocol was adapted from Rosen et al. (Nature Chemistry, 6, 2014, 804-809).

To a solution of DNA (10 nmol) in water (50 μL), MeCN (50 μL) and Et₃N (1 μL) Bis(2,5-dioxopyrrolidin-1-yl) octanedioate in DMF (50 μM, 50 μL) was added. The solution was shaken at rt for 30 min. The DNA was ethanol precipitated by addition of aq. NaOAc (3 M, 18 μL, pH=5.2), cold ethanol (500 μL) and glycogen (1 μL, 20 mg/mL). The solution was incubated in liq. N₂ for 1 min followed by cold centrifugation for 45 min (4° C., 20000 g). The supernatant was removed, and the pellet dissolved in 200 μL TEAA buffer (0.1 M) and subjected to purification by RP-HPLC 10-20% MeCN in 0.1 TEAA over 15 min. T=25° C., flow rate=1 mL/min. The fractions containing product was pooled and an equivalent volume of 2% aq. TFA solution was added. The mixture was aliquoted, lyophilized and stored at −20° C. Rt=9.8 min (24%).

Guiding Strand (Dabigatran) (11) and Analyte Analogue (Dabigatran) (6):

DNA (10 nmol) in water (5 μL) was added to a solution of carbonate buffer (200 μL, 20 mM, pH 8.5), water (5 μL), dabigatran (50 μL, 7.6 mM in DMSO with 10% 0.1 M HCl) and freshly prepared DMTMMCI (40 μL, 0.5 M in H₂O). The mixture was shaken for 2 h at 25° C. Ethanol precipitation was performed by addition of aq. NaOAc (3 M, 32 μL, pH=5.2), cold EtOH (990 μL) and glycogen (1 μL, 20 mg/mL). The mixture was cooled in liq. N₂ followed by cold centrifugation for 45 min (4° C., 20000 g). The supernatant was removed, and the pellet dissolved in 0.1 M TEAA buffer, which was subjected to purification by RP-HPLC 5-20% MeCN in 0.1 TEAA over 30 min. T=25° C., flow rate=1 mL/min.

Dabigatran guiding strand: Rt=19.9 min, LCMS: [M]; Calc.: 8026, found: 8026. Dabigatran acceptor strand: HPLC 10-35% MeCN in TEAA over 15 min. Rt=10.1 min, LCMS: [M]; Calc.: 11426, found: 11426.

Generic Protein Labelling Protocol (Analyte Binding Protein (3))

The protein (0.25 μM, 1 eq.) was added to a solution of the reactive strand (13) (0.3 μM, 1.2 eq.) and guiding strand (11) (0.3 μM, 1.2 eq.) in HEPES buffer (50 mM, pH 8.0) and NaCl (400 mM). The reaction mixture was incubated overnight at rt. The releasing strand (12) (0.6 μM, 2 eq.) was added, and the reaction mixture incubated for 30 min at rt. The mixture was concentrated by Amicon Ultra® centrifugal filters (MWCO 30K, 14100 g for 10 min) and purified by anion exchange HPLC. Anion exchange was performed with a Thermo Scientific™ Dionex™ DNAPac™ PA-100 4×250 mm column on a Hewlett Packard Agilent 1100 Series HPLC system. Purification was performed with Buffer A (25 mM Tris) and Buffer B (25 mM Tris, 1 M NaCl) using an increasing gradient of buffer B (0-75%) over 10 min (flow rate: 1 mL/min, T=25° C.). Fractions containing the analyte binding protein (3) were collected and washed twice with HEPES buffer (10 mM, pH 7.0, 0.02 v/v % Tween®-20) in Amicon Ultra® centrifugal filters (MWCO 30K, 14100 g for 30 min). The concentration of product was determined by the Cy3 absorbance at 550 nm. For the dabigatran assay, idarucizumab was used as antibody.

Rt (analyte binding protein (3) for dabigatran)=9.5 min

Assay Setup

In a typical assay, the analyte binding protein (3) conjugate and analyte analogue (6) are kept at a 1:1 stoichiometry. The conjugate (50 nM) was mixed with the analyte (1) dabigatran (500 nM) in either HEPES (50 mM, pH 7.0) or human plasma and incubated at room temperature for 5 min. The analyte analogue (6) (50 nM) was added and the FRET measurements were initiated.

FRET Measurements

To a quartz cuvette, that is washed 3 times with MilliQ water between samples, was added 70 μL of the assay solution. Excitation of the Cy3 dye was performed at 530 nm, and the spectrum was measured from 540-750 nm. A background measurement without analyte binding protein (3) conjugate and analyte analogue (6) was subtracted from the measured spectra. FRET was calculated as E=I_{A}/(I_{A}+I_{D}), where the I_{A} was measured at 650 nm for Cy5 and I_{D} was measured at 550 nm for Cy3.

For kinetic measurements, the analyte analogue (6) was added directly to the cuvette containing the remaining assay solution, and the FRET measurements were initiated with timepoints every 45 sec for 10 min. For temperature measurements, the cuvette containing the assay solution without the acceptor strand was incubated for 10 min at the investigated temperature.

Results

The binding of the analyte binding protein (3) to the analyte analogue (6) brings the Cy3 dye on the conjugate in close proximity to the Cy5 dye on the analyte analogue (6), which is measured as a high FRET signal. However, when analyte (1) dabigatran is present in the assay it will block the binding of the acceptor strands, which results in a decrease in the FRET signal compared to a sample without dabigatran present. Since the decrease in the FRET signal is dependent on the concentration of dabigatran, a titration curve of known concentration of dabigatran was constructed (FIG. 5A) for the determination of the dabigatran in samples with unknown concentrations. The measurements could be performed reliably in buffer. The assay was capable of detecting the presence and absence of the analyte (1) dabigatran in 10 min (FIG. 5B) in 87% plasma. The kinetic measurements in 87% plasma show that the assay has reach the maximum signal after only 2-3 mins at 25° C. and that in the presence of dabigatran no time dependency is observed (FIGS. 5C and 5D), but the kinetics are significantly slower at lower temperatures, which is believed to be caused by the decreased diffusion of the components due to the increased viscosity of the sample.

### CONCLUSION

In a conventional fluorometer and cuvette setup the assay is able to rapidly measure the analyte up to 500 nM in both buffer and plasma. The important threshold for clinical decision making is 74 nM, which is within the dynamic range of the reported assay.

### Example 4—Detection of Dabigatran in a Lateral Flow Chip

The aim of these experiments was to detect the presence/concentration of the anticoagulant dabigatran at 74 nM in human plasma (and other complex matrices) and whole blood by measuring FRET using a LED as excitation source in the lateral flow chip (FIG. 3).

Materials:

The strands and conjugate from example 3 were used for these experiments.

Methods:

Solid support (7) preparation.

Fusion 5 filter papers (23×6 mm for plasma, 40×6 mm for whole blood) were spotted with 1 pmol of analyte binding protein (3) at an 8 mm distance from the end and 1 pmol analyte analogue (6) was spotted at a 2 mm distance from the same end. Before use, the solid support (7) were stored under reduced pressure overnight after addition of the biomolecules.

Chip Setup

A small strip of prepared filter paper containing the biomolecules is placed in the cartridge shown in FIG. 3. The chip was placed in the instrument with a LED light source. At the end of the fusion 5 filter paper furthest from the detection chamber (10) was added sample (100 μL for human plasma, milk, beer, saliva, urine or 400 μL for whole blood), and the FRET measurements were started. In general, the FRET of the assays were measured over 5/10 min (plasma, milk, beer, saliva, urine/whole blood, respectively), where an average of data points for the last 100 seconds were used for the analysis, since the signals were stable at these time points.

Results

The chips loaded with 1 pmol analyte binding protein (3) and 1 pmol analyte analogue (6) were titrated with varying concentrations of dabigatran (0-100 ng/mL) in human plasma/whole blood (FIG. 6A). The performance was likewise tested against other complex matrices such as milk, beer, saliva and urine and showed a clearly distinction between presence and absence of 1 μM dabigatran in 5 min (FIG. 6B). The described method was lastly tested against the conventional method for dabigatran detection HEMOCLOT®. The setup shows similar performance as the conventional method (FIG. 6C).

### CONCLUSION

The detection chip (10) fitted with a solid support (7) containing the reagents for the dabigatran assay. The system readily detects the analyte in various complex matrices and is performing similar to the conventional standard for dabigatran measurements.

### Example 5—Detection of Apixaban in Spectrofluorometer Setup

Aim of Study

The aim of these experiments was to detect the presence/concentration of the anticoagulant apixaban at 54.4 nM in human plasma by measuring FRET using a spectrofluorometer.

Materials:

An analyte binding protein (3) and an analyte analogue (6) is used for this experiment. The DNA-strands are modified with internal Cy3 or Cy5 fluorophores and contain 3′-amino modifier for the functionalization with a carboxylic acid containing analogue of apixaban.

Strand Sequences:

The starting material strands were the same as used in example 3 and were further modified according to the protocols in the methods section.

The strands were purchased from Integrated DNA technologies. In general, the chemicals were purchased from Sigma-Aldrich. An apixaban analogue containing a carboxylic acid in place of the primary amide was purchased at Acesys Pharmatech. The anti-apixaban antibody 79-2 was developed and provided by BioPorto A/S.

Methods:

Modification of DNA Strands:

The reactive strand (13) for protein conjugate formation was the same as used in example 3.

Preparation of Apixaban Modified Analyte Analogue (6) and Guiding Strand (11):

To a solution of DNA (20 nmol) in water (10 μL) was added carbonate buffer (200 μL, 20 mM, pH 8.5), water (30 μL), acid modified apixaban derivative (20 μL, 10 mg/mL in DMSO) and freshly prepared DMTMMCI (40 μL, 0.5 M in H₂O). The mixture was shaken at 25° C. for 2 h. The DNA was precipitated by addition of aq. NaOAc (3 M, 32 μL, pH=5.2), cold EtOH (990 μL) and glycogen (1 μL, 20 mg/mL). The mixture was cooled in liq. N₂ followed by centrifugation for 45 min (4° C., 20000 g). The supernatant was removed, and the pellet dissolved in 0.1 TEAA buffer, which was subjected to purification by RP-HPLC 10-20% MeCN in 0.1 TEAA over 15 min. T=25° C., flow rate=1 mL/min.

Apixaban guiding strand (11): Rt=10.8 min, LCMS: [M]; Calc.: 8015, found: 8016.

Apixaban analyte analogue (6): Rt=12.6 min, LCMS: [M]; Calc.: 11414, found: 11416.

Antibody Conjugate

The antibody conjugate of the anti-apixaban antibody, 79-2, was prepared by the generic protein conjugation protocol from example 3.

Rt (analyte binding protein (3) for apixaban)=9.7 min

Assay Setup

In a typical assay, the analyte binding protein (DNA(Cy3)-anti apixaban antibody conjugate) (3), and the analyte analogue (apixaban) (6) is kept at a 1:2.5 stoichiometry. The conjugate (25 nM) was mixed with apixaban (0-1000 nM) in human plasma and incubated at room 5-45° C. for 10 min. The analyte analogue (6) (63 nM) was added and the FRET measurements were initiated.

FRET Measurements

To a quartz cuvette, that is washed 3 times with MilliQ water between samples, was added 70 μL of the assay solution. Excitation of the Cy3 dye was performed at 530 nm, and the spectrum was measured from 540-750 nm. A background measurement without DNA-protein conjugate and acceptor strand was subtracted from the measured spectra. FRET was calculated as E=I_{A}/(I_{A}+I_{D}), where the I_{A} was measured at 650 nm for Cy5 and I_{D} was measured at 550 nm for Cy3. For kinetic measurements, the acceptor strand was added directly to the cuvette containing the remaining assay solution, and the FRET measurements were initiated with timepoints every 45 sec for 10 min. For temperature measurements, the cuvette containing the assay solution without the acceptor strand was incubated for 10 min at the investigated temperature.

Results

The binding of the antibody binding protein (3) (DNA-anti apixaban antibody) to the analyte analogue (apixaban) (6) was evaluated by FRET measurements (FIG. 7). The measurements show an increased FRET signal upon binding. This interaction could be prevented or partially prevented by first incubating the protein conjugate with either high concentrations of free apixaban (1 μM) or the therapeutically relevant concentration 54.4 nM before addition of the acceptor. This resulted in a concentration dependent decrease in the FRET signal. Kinetic measurements of the assay reached maximum signal after 2-3 mins at 25° C. (FIGS. 7A and 7B). Furthermore, measurements performed at 5-45° C. were performed. The assay showed reliable performance from 5-45° C. as both presence and absence of analyte (6) resulted in a similar increase of signal. The kinetics of the assay is lowered at 5° C., which is believed to be an effect of the decreased diffusion of the components due to the increased viscosity of the sample.

### CONCLUSION

The assay is able to rapidly distinguish the presence and absence of the small molecule apixaban.

### Example 6—Detection of Apixaban in Lateral Flow Chip

The aim of these experiments was to detect the presence/concentration of the anticoagulant apixaban at 54.4 nM (threshold concentration used at hospitals) in human plasma by measuring FRET using an LED as excitation source in the chip (FIG. 3).

Materials:

The strands and conjugate from example 5 was used for these experiments.

Methods:

Solid Support (7) Preparation.

Fusion 5 filter papers (23×6 mm) were spotted with 0.5 pmol of analyte binding protein (3) at an 8 mm distance from the detection chamber and 1.25 pmol analyte analogue was spotted at a 2 mm distance from the same end. The analyte binding protein (3) was spotted in a 5/5% sucrose/trehalose buffer whereas the analyte analogue (6) was spotted in water. The solid support (7) was stored under reduced pressure for 1 hour.

Chip Setup

A small strip of prepared solid support (7) (23×6 mm) containing the assay reagents is placed in the cartridge according to FIG. 3. The chip was placed in an instrument with a light source, optical filters, and detectors that monitors the emitted light at two wavelengths. At the end of the solid support (7), furthest from the readout window, was added 100 μL of human plasma and the FRET measurements were started. In general, the FRET of the assays were measured over 10 min, where an average of data points for the last 100 seconds were used for the analysis, since the signals were stable at these time points.

Results

The chips loaded with 0.5 pmol analyte binding protein (3) and 1.25 pmol analyte analogue (6) were titrated with varying concentrations of apixaban (0-500 nM) in human plasma (FIG. 7C). A change in the FRET signals were observed when apixaban was present in the plasma, and the results indicate that apixaban can be detected at 50 nM in human plasma within only 10 min.

### CONCLUSION

The apixaban assay in the detection chip (10) allows for detection of apixaban in the range 0-500 nM which gives rise to a linear response.

### Example 7—Detection of Linezolid in Spectrofluorometer Setup

The aim of these experiments was to detect the presence/concentration of the antibiotic linezolid at the nanomolar range in human plasma by measuring FRET using a spectrofluorometer and testing the stability of the assay at different temperatures.

Materials:

An analyte binding protein (3) and an analyte analogue (6) are used for this experiment. The DNA-strands are modified with internal Cy3 or Cy5 fluorophores and contain 3′-amino modifiers for the functionalization with an analogue of linezolid containing an amine in place of the amide. The functionalization is performed in a sequential manner.

Strand Sequences:

The starting material strands were the same as in example 3. These strands were modified according to the protocols in the methods section below.

The strands were purchased from Integrated DNA technologies. In general, the chemicals were purchased from Sigma-Aldrich. A linezolid analogue containing an amine in place of the amide was purchased at Matrix Scientific. The anti-linezolid antibody 74-6 was developed and provided by BioPorto A/S.

Methods:

Modification of DNA Strands:

The reactive strand (13) for protein conjugate formation was the same as used in example 3.

The Analyte Analogue (6) and Guiding Strand (11) for Protein Conjugate Formation:

To a solution of DNA (10 μL, 200 μM) in sodium carbonate buffer (60 μL, 20 mM, pH 8.5) was added acetonitrile (40 μL) and bis-NHS-ester linker (disuccinimidyl glutarate) (20 μL, 10 mg/mL in DMF). The reaction mixture was incubated at 25° C. for 30 minutes followed by ethanol precipitation. The pellet was redissolved in sodium carbonate buffer (50 μL, 20 mM, pH 8.5) and a solution of Linezolid amine analogue (25 μL, 5 mg/mL in DMF) was added and the mixture was incubated at 25° C. for 2 h followed by ethanol precipitation and purification by RP-HPLC 10-35% MeCN in 0.1 TEAA over 15 min. T=25° C., flow rate=1 mL/min. Linezolid guiding strand: Rt=7.4 min, LCMS [M]; Calc.: 7963.1, found: 7963.4 Linezolid acceptor strand: Rt=11.3 min, LCMS [M]; Calc.: 11362.9, found: 11363.8

Antibody Conjugate

The analyte binding protein (3) (anti-linezolid antibody, 74-6), was prepared by the generic protein conjugation protocol from example 3. Two different analyte binding proteins (3) were prepared; one protein containing a dye (5A) close to one of the two paratopes (4) (termed single-modified protein) and one protein containing dyes (5A) close to both of the two paratopes (4) (termed double-modified protein).

Rt (linezolid single-modified binding protein)=9.6 min

Rt (linezolid double-modified binding protein)=10.5 min

Assay Setup

In a typical assay, the analyte binding protein (3) and analyte analogue (6) is kept at a 1:2.5 stoichiometry. The conjugate (25 nM) was mixed with linezolid (0-1000 nM) in human plasma and incubated at room 5-45° C. for 10 min. The acceptor strand (63 nM) was added and the FRET measurements were initiated.

FRET Measurements

To a quartz cuvette that is washed 3 times with MilliQ water between samples, was added 80 μL of the assay solution. Excitation of the Cy3 dye was performed at 530 nm, and the spectrum was measured from 540-750 nm. A background measurement without DNA-protein conjugate and acceptor strand was subtracted from the measured spectra. FRET was calculated as E=I_{A}/(I_{A}+I_{D}), where the I_{A} was measured at 650 nm for Cy5 and I_{D} was measured at 550 nm for Cy3. For kinetic measurements, the acceptor strand was added directly to the cuvette containing the remaining assay solution, and the FRET measurements were initiated with timepoints every 45 sec for 9 min. For temperature measurements, the cuvette containing the assay solution without the acceptor strand was incubated for 10 min at the investigated temperature prior to addition of the acceptor strand.

Results

FRET measurements were performed to evaluate if the DNA-anti-linezolid antibody could bind the linezolid modified acceptor strand. The measurements showed high FRET signals, when the components were mixed together. Upon binding addition of small molecule linezolid prior to addition of the acceptor strand, a concentration dependent decrease in the FRET signal was observed. Thereby, it was possible to measure the linezolid.

The assay was evaluated by kinetic measurements at 5-45° C. The measurements at 25° C. demonstrated, the assay reached a maximum signal after only 2-3 min (FIGS. 7A and 7C, for single-modified protein and double-modified protein, respectively). In the presence of linezolid no signal developed over time (FIGS. 7B and 7D, for single-modified protein and double-modified protein, respectively). The assay showed good performance up to 25° C., yet the signal was decreasing at increasing temperature. The kinetics were slower at 5° C. due to lowered diffusion of the components in the cold plasma.

### CONCLUSION

The linezolid assay (both single-modified protein and double-modified protein) performs well in 87/79% human plasma (for single-modified protein/double-modified protein, respectively), and allows for rapid detection of linezolid.

### Example 8—Detection of Linezolid in a Lateral Flow Chip

The aim of these experiments was to detect the presence/concentration of the antibiotic linezolid at 50 nM in human plasma by measuring FRET using a LED as excitation source in the lateral flow chip (FIG. 3).

Materials:

The strands and conjugate (only double-modified protein) from example 7 were used for these experiments.

Methods:

Solid Support (7) Preparation.

Fusion 5 filter papers (23×6 mm for plasma, 40×6 mm for whole blood) were spotted with 0.5 pmol of analyte binding protein (3) at an 8 mm distance from the end and 1.25 pmol analyte analogue (6) was spotted at a 2 mm distance from the same end. Before use, the solid support (7) were stored under reduced pressure overnight after addition of the biomolecules.

Chip Setup

A small strip of prepared filter paper containing the biomolecules (23×6 mm) is placed in the cartridge shown in FIG. 3. The chip was placed in an instrument with a light source, optical filters, and detectors that monitors the emitted light at two wavelengths. At the end of the fusion 5 filter paper furthest from the readout window was added 100 μL of human plasma or 400 μL whole blood, and the FRET measurements were started. In general, the FRET of the assays were measured over 10 min, where an average of data points for the last 100 seconds were used for the analysis, since the signals were stable at these time points.

Results

The chips loaded with 0.5 pmol analyte binding protein (3) and 1.25 pmol analyte analogue (6) were titrated with varying concentrations of linezolid (0-1000 nM) in human plasma (FIG. 7C). A change in the FRET signals was observed when linezolid was present in the plasma, and the results indicate that linezolid can be detected at the nanomolar range in human plasma within only 10 min.

### CONCLUSION

The detection chip (10) fitted with a solid support (7) containing the reagents for the linezolid assay allowed for detection of linezolid in the nanomolar range both in 93% plasma and 93% whole blood.

### Example 9—Unspecific Labelled Proteins in a Dabigatran Competition Assay on a Fluorometer Setup

The aim of these experiments was to quantify the performance of unspecific labelled protein conjugates in buffer in a fluorometer setup.

Materials:

The starting material strands were the same as used in Example 3 and were further modified according to the protocols in the methods section Methods:

An analyte binding protein (3), a protein covalently linked to the first member (5A) of the optical pair (5) in a unspecific manner (Global Cy3 Conjugate (Glo-Cy3), a protein covalently linked to a DNA strand containing first member (5A) of the optical pair (5) in a unspecific manner (Glo-DNA), and an analyte analogue (6) is used for this experiment. The DNA-strands are modified with internal Cy3 or Cy5 fluorophores and contain 3′-amino-modifier for the functionalization with dabigatran.

Modification of DNA Strands:

Preparation of the DBCO Strand

Dibenzocyclooctyne-N-hydroxysuccinimidyl (DBCO) ester in DMF (100 μL, 20 mM) was added to a solution of DNA (20 nmol) in water (100 μL) and Et₃N (1 μL). The solution was shaken overnight at rt. Ethanol precipitation of the DNA was performed by addition of aq. NaOAc (3 M, 28 μL, pH=5.2), cold ethanol (502) and glycogen (1 μL, 20 mg/mL). The solution was incubated in liq. N₂ for 1 min followed by centrifugation for 45 min (4° C., 20000 g). The supernatant was removed immediately after, and the pellet was dissolved in 200 μL MilliQ and subjected to purification by RP-HPLC 10-20% MeCN in 0.1 M TEAA over 15 min. T=25° C., flow rate=1 mL/min. The fractions containing product was pooled. DBCO-donor strand: Rt=12.4 min (24%), LCMS MS [M]; Calc.: 7655.4, found: 7652.2

Antibody Conjugate

Preparation of Global DNA Conjugate (Glo-DNA)

To a solution of the NHS azide (2,5-dioxopyrrolidin-1-yl 3-(2-(2-(2-azidoethoxy)ethoxy)ethoxy)propanoate) (25 μM, 5 eq.) in HEPES buffer (50 mM, pH 8.0) was added the antibody (5 μM, 1 eq.). The reaction mixture was incubated at rt overnight. The reaction mixture concentrated by Amicon Ultra® centrifugal filters (MWCO 3K, 14100 g for 30 min) and washed thrice with HEPES buffer (50 mM, pH 8.0). The azide modified antibody was used without further purification.

To a solution of the DBCO strand (5 μM, 1 eq.) in HEPES buffer (50 mM, pH 8.0) was added the azide modified antibody (25 μM, 5 eq.). The reaction mixture was incubated at rt overnight. The reaction was purified by anion exchange HPLC. Anion exchange was performed with a Thermo Scientific™ Dionex™ DNAPac™ PA-100 4×250 mm column on a Hewlett Packard Agilent 1100 Series HPLC system. The purification was performed with Buffer A (25 mM Tris) and Buffer B (25 mM Tris, 1 M NaCl) with an increasing gradient of buffer B (0-75%) over 10 min (flow rate: 1 mL/min, T=25° C.). Fractions containing the conjugates were collected and washed twice with HEPES buffer (10 mM, pH 7.0, 0.02 v/v % Tween®-20) in Amicon Ultra® centrifugal filters (MWCO 3K, 14100 g for 30 min) to remove the majority of the salt from the samples. DNA-protein conjugate Rt=10.4 min. The concentration of the conjugates was determined using the Cy3 absorbance at 550 nm.

Preparation of Global Cy3 Conjugate (Glo-Cy3)

To a solution of the NHS-Cy3 (3H-Indolium, 2-[3-(1,3-dihydro-1,3,3-trimethyl-2H-indol-2-ylidene)-1-propen-1-yl]-1-[6-[(2,5-dioxo-1-pyrrolidinyl)oxy]-6-oxohexyl]-3,3-dimethyl-, tetrafluoroborate) (100 μM, 5 eq.) in DMSO was added the antibody (20 μM, 1 eq.) in HEPES buffer (50 mM, pH 8.0). The final DMSO concentration was 5%. The reaction mixture was incubated at rt overnight. The reaction mixture concentrated by Amicon Ultra® centrifugal filters (MWCO 3K, 14100 g for 30 min) and washed thrice with HEPES buffer (50 mM, pH 8.0). The protein was used without further purification. The concentration of the protein was determined using the absorbance at 280 nm and the concentration of Cy3 using the absorbance at 550 nm. This yielded an antibody to Cy3 ratio of 1.6.

Assay Setup

In a typical assay, the analyte binding protein (3) conjugate and analyte analogue (6) are kept at a 1:1 stoichiometry. The conjugate (25 nM) was mixed with either the analyte (1) dabigatran (250 nM) or the analyte analogue (6) in HEPES (50 mM, pH 7.0) incubated at room temperature for 10 min. The analyte analogue (6) (25 nM) or the analyte (250 nM) was added and the FRET measurements were initiated.

FRET Measurements

To a quartz cuvette, that is washed 3 times with MilliQ water between samples, was added 70 μL of the assay solution. Excitation of the Cy3 dye was performed at 530 nm, and the spectrum was measured from 540-750 nm. A background measurement without analyte binding protein (3) conjugate and analyte analogue (6) was subtracted from the measured spectra. FRET was calculated as E=I_{A}/(I_{A}+I_{D}), where the I_{A} was measured at 650 nm for Cy5 and I_{D} was measured at 550 nm for Cy3.

For kinetic measurements, the analyte analogue (6)/dabigatran was added directly to the cuvette containing the remaining assay solution, and the FRET measurements were initiated with timepoints every 2 min for 10 min.

Results

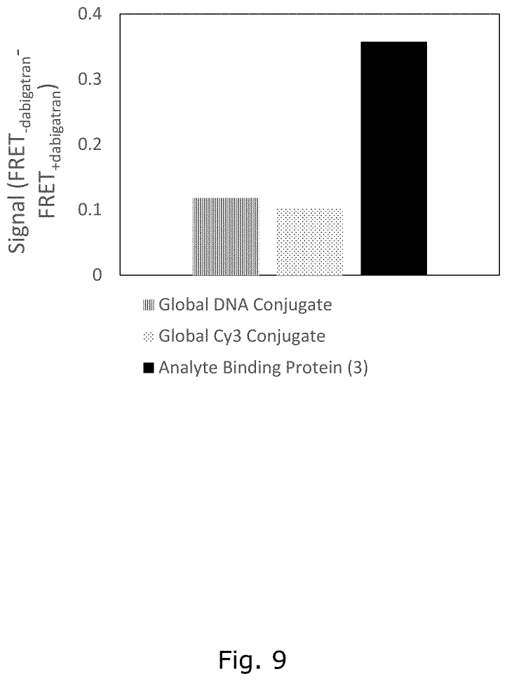

The binding of the analyte binding protein (3) to the analyte analogue (6) brings the Cy3 dye on the conjugate in close proximity to the Cy5 dye on the analyte analogue (6), which is measured as a high FRET signal. However, when analyte (1) dabigatran is present in the assay it will block the binding of the acceptor strands, which results in a decrease in the FRET signal compared to a sample without dabigatran present. The results are shown in FIG. 9. In the case of the Glo-Cy3 and Glo-DNA, the signal (FRET at 0 μM dabigatran minus FRET at 250 μM dabigatran) is significantly lower for the unspecifically labelled proteins than the signal of the specifically labelled assay described in Example 3. For the case of Glo-Cy3, this reduction in signal is expected to be due to a high amount of aggregation of the modified protein over time. For the case of Glo-DNA, the reduction is believed to be a result in the lower proximity, as a global labelled protein would have greater interdistance of the optical pair (5) than the specific conjugate.

### CONCLUSION

In a conventional fluorometer and cuvette setup, the global labelled controls performed significant worse than the assay described in Example 3.

### Example 10—Dabigatran Assay and Controls in a Lateral Flow Chip

The aim of these experiments was to quantify the performance of the unspecific conjugates (Glo-Cy3 and Glo-DNA) in human plasma by measuring FRET using a LED as excitation source in the lateral flow chip (FIG. 3).

Materials:

The strands and conjugate from Example 9 were used for these experiments.

Methods:

Solid Support (7) Preparation

Fusion 5 filter papers (40×6 mm for plasma) were spotted with 1 pmol of analyte binding protein (3)/Glo-Cy3/Glo-DNA at an 8 mm distance from the end and 1 pmol analyte analogue (6) was spotted at a 2 mm distance from the same end. Before use, the solid support (7) were stored under reduced pressure overnight after addition of the biomolecules.

Chip Setup

A small strip of prepared filter paper containing the biomolecules is placed in the cartridge shown in FIG. 3. The chip was placed in the instrument with a LED light source. At the end of the fusion 5 filter paper furthest from the detection chamber (10) was added plasma (200 μL) and the FRET measurements were started. In general, the FRET of the assays was measured over 10 min. where an average of data points for the last 100 seconds was used for the analysis, since the signals were stable at these time points.

Results

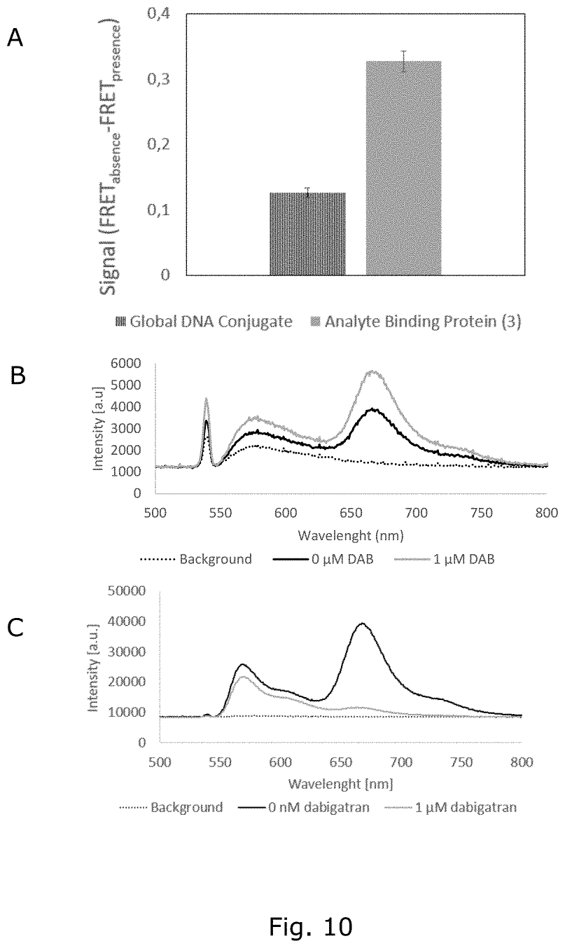

The FRET in presence and absence of 1 μM dabigatran was measured and the data is shown in FIG. 10A. Comparing the analyte binding protein (3) and Glo-DNA, it is evident that the signal (FRET in absence minus FRET in presence of dabigatran) is significantly larger using analyte binding protein compared to Glo-DNA. This result is expected based on findings of Example 9 shown in FIG. 9. The raw intensity for Glo-Cy3 in presence and absence of 1 μM dabigatran is found in FIG. 10B. The Glo-Cy3 did not perform at all in the chips. This is expected to be due to aggregation of Glo-Cy3 on the paper, which retards the migration of compounds into the detection chamber (10). Only the analyte analogue (6) migrates to the window, as seen from the distinct peak. On FIG. 10C the performance of analyte binding protein (3) (dabigatran) assay is shown. The assay performs well and the distinct peaks for the optical pair (5) is shown in FIG. 10C. The signal is likewise significantly greater than the background.

### CONCLUSION

The unspecific labelled proteins, Glo-Cy3 and Glo-DNA, do not perform well in the lateral flow chip. The FRET signal is either low or no signal from the protein conjugate is present in the detection chamber (10).

### Example 11—Competition Experiment of the Dabigatran Assay on a Fluorometer Setup

The aim of these experiments was to investigate to what extend outcompetition between analyte binding protein (3), analyte analogue (6) (dabigatran) and dabigatran is occurring. The aim is likewise to investigate whether or not the order of addition of the assay species is crucial. Phrased in another way the aim is likewise to investigate the effect of the order of addition of the assay species.

Materials:

An analyte binding protein (3) (dabigatran) and an analyte analogue (6) (dabigatran) is used for this experiment. The strands and conjugate from Example 3 were used for these experiments.

Methods:

Assay Setup

In a typical assay, the analyte binding protein (3), and the analyte analogue (dabigatran) (6) is kept at a 1:1 stoichiometry. The conjugate (25 nM) was mixed with either dabigatran (250 nM, 25 nM, 0 nM) or analyte analogue (6) (25 nM) in HEPES pH 7.5 and incubated at rt for 10 min. To the sample lacking analyte analogue (6) was added analyte analogue (6), and to the sample lacking dabigatran was added dabigatran. Hereafter the FRET measurements were initiated.

FRET Measurements

To a quartz cuvette, that is washed 3 times with MilliQ water between samples, was added 70 μL of the assay solution. Excitation of the Cy3 dye was performed at 530 nm, and the spectrum was measured from 540-750 nm. A background measurement without DNA-protein conjugate and acceptor strand was subtracted from the measured spectra. FRET was calculated as E=I_{A}/(I_{A}+I_{D}), where the I_{A} was measured at 650 nm for Cy5 and I_{D} was measured at 550 nm for Cy3. For kinetic measurements, the FRET measurements were initiated with timepoints every 2 min for 10 min.

Results

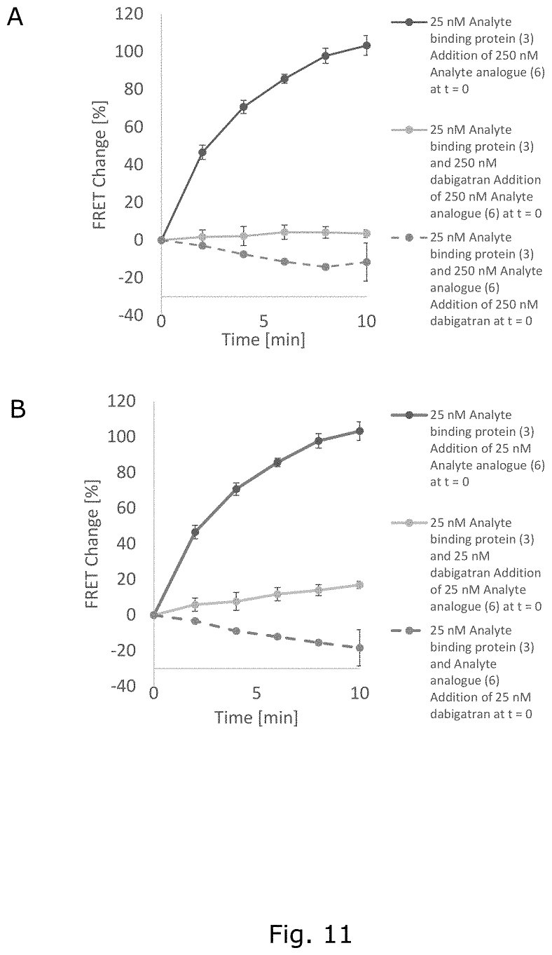

The binding of the antibody binding protein (3) to the analyte analogue (6) was evaluated by FRET measurements (FIG. 11). The signal is plotted as the ‘FRET change’ being the percentile difference from the initial data point (0 minutes). A) The measurements show an increased FRET signal upon binding between the antibody binding protein (3) to the analyte analogue (6) in the absence of dabigatran. If the protein has been incubated for 10 minutes with 10 eq. dabigatran prior to the addition of the analyte analogue (6), only limited signal is obtained over 10 minutes. If the antibody binding protein (3) and the analyte analogue (6) is incubated for 10 minutes prior to addition of 10 eq. dabigatran, only limited outcompetition between analyte analogue (6) and dabigatran is observed. B) When performing the competition experiment described in A) using 1 eq. dabigatran, a slightly larger signal is generated in both cases, yet the signal is still minor in comparison to the un-inhibited setup.

### CONCLUSION

Outcompeting the binding of analyte binding protein (3) and analyte analogue (6) is only limited in presence of both 1 and 10 eq. of dabigatran. This concludes that the order of addition of the assay species is important. Thus, the order of addition of the assay species influences the efficiency of the assay.

1. A method for determining the presence and/or concentration of an analyte in a sample, said method comprising:
I. providing a sample to be analyzed for the presence of the analyte;
II. providing an analyte binding protein, said analyte binding protein comprising:
a binding site for the analyte; and
a first member of an optical signal pair covalently coupled to the analyte binding protein through a first oligonucleotide linker;
III. providing an analyte analogue, said analyte analogue being covalently coupled to a second member of the optical signal pair through a second oligonucleotide linker;
wherein, when said analyte binding protein and said analyte analogue are in proximity, an optical signal is generated which is different from the optical signal generated when the analyte binding protein and the analyte analogue are not in proximity;
IV. bringing in contact said sample, said analyte binding protein and said analyte analogue, and
V. determining the presence or absence of the analyte in said sample by measuring an optical signal;
and/or
VI. determining the concentration of the analyte in said sample by measuring an optical signal.

2-26. (canceled)

27. The method according to claim 1, wherein the first oligonucleotide linker is an oligonucleotide consisting of between 2 and 100 nucleotides.

28. The method according to claim 1, wherein the first oligonucleotide-linker and the second oligonucleotide linker are non-complementary.

29. The method according to claim 1, wherein the first oligonucleotide-linker and the second oligonucleotide linker are DNA linkers.

30. The method according to claim 1, wherein the concentration of the analyte in the sample is determined.

31. The method according to claim 1, wherein, in said determination step V.,
when said generated optical signal is different from a reference optical signal, it is indicative of the presence of said analyte in said sample; or
when said generated optical signal is not different from a reference optical signal, it is indicative of the absence of the analyte in said sample.

32. The method according to claim 1, wherein the distance between the first member and the second member, when the analyte analogue is bound to the analyte binding protein, is in the range 1-100 Ångstrom.

33. The method according to claim 1, wherein said sample is a biological sample selected from the group consisting of blood, blood plasma, blood serum, saliva, urine, CSF, a tissue sample, a water sample, an environmental sample, a food sample, a beverage, a surface swap, a medical formulation, a drug formulation, an addictive substance, and an addictive formulation.

34. The method according to claim 1, wherein said analyte is selected from the group consisting of a small molecule, a peptide and a protein.

35. The method according to claim 1, wherein the analyte is an anticoagulant.

36. The method according to claim 1, wherein said sample is brought in contact with the analyte binding protein before being brought in contact with said analyte analogue.

37. A solid support comprising:
a) a first region comprising an analyte binding protein, said analyte binding protein comprising:
a binding site for an analyte; and
a first member of an optical signal pair covalently coupled to the analyte binding protein through a first oligonucleotide linker; and
b) a second region comprising an analyte analogue, said analyte analogue being covalently coupled to a second member of the optical signal pair through a second oligonucleotide linker.

38. The solid support according to claim 37, wherein said first region and said second region are at independent regions at the solid support.

39. The solid support according to claim 37, wherein the first region and the second region are arranged in serial connection on the solid support.

40. The solid support according to claim 37, wherein the solid support comprises an anterior end comprising the first region and a posterior end comprising the second region.

41. The solid support according to claim 37, wherein the solid support comprises an anterior end comprising the first region and a posterior end comprising the second region; and
wherein the anterior region comprises an inlet for receiving the sample.

42. The solid support according to claim 37, wherein the solid support is porous.

43. The solid support according to claim 37, being mounted in a cartridge.

44. A kit comprising:
I. a first vial comprising an analyte binding protein, said analyte binding protein comprising:
a binding site for an analyte; and
a first member of an optical signal pair covalently coupled to the analyte binding protein through a first oligonucleotide linker;
II. a second vial comprising an analyte analogue, said analyte analogue being covalently coupled to a second member of the optical signal pair through a second oligonucleotide linker;
III. optionally, a solid support material for depositing the content of the first vial and the second vial at different distinct regions; and
IV. optionally, a cartridge for mounting the solid support material.

45. The kit according to claim 44, further comprising:
III. the solid support material for depositing the content of the first vial and the second vial at different distinct regions.


SEQUENCE LISTING (4 sequences)

SEQ ID NO: 1 - DNA, 24 bp, Artificial Sequence
  reactive strand
  misc_feature (20)..(20): Cy3 linked spacer
  misc_feature (24)..(24): 3′ Amino modifier
acatacagcctcgcatgagncccn

SEQ ID NO: 2 - DNA, 25 bp, Artificial Sequence
  Guiding strand
  misc_feature (1)..(1): 5′ Amino modifier C6
ngggtctcatgcgaggcttacgaac

SEQ ID NO: 3 - DNA, 38 bp, Artificial Sequence
  Release strand
gttcgtaagcctcgcatgagacccgtaggcaatcctac

SEQ ID NO: 4 - DNA, 36 bp, Artificial Sequence
  Analyte analog
  misc_feature (32)..(32): Cy5 linked spacer
  misc_feature (36)..(36): 3′ Amino modifier
tttcacacttccttactgagtctatctattcnaccn